(19) United States
(12) Patent Application Publication
(10) Pub. No.: US 2005/0256065 A1
(43) Pub. Date: Nov. 17, 2005

(54) Method for stabilizing changes in corneal curvature in an eye by administering compositions containing stabilizing ophthalmic agents

(75) Inventors: Donald H. Harris, Newport Beach, CA (US); Charles Harrison May, San Diego, CA (US); Stuart Grant, Los Angeles, CA (US); Patricia Zuk, Marina del Rey, CA (US)

Correspondence Address: MANDEL & ADRIANO, 55 SOUTH LAKE AVENUE, SUITE 710, PASADENA, CA, 91101, US

(73) Assignee: Permasight, Newport Beach, CA (US)

(21) Appl. No.: 11/046,507

(22) Filed: Jan. 26, 2005

Related U.S. Application Data
(60) Provisional application No. 60/539,461, filed on Jan. 26, 2004.

(51) Int. Cl. A61K 31/704; A61K 31/573

(52) U.S. Cl. 514/37; 514/171

(57) ABSTRACT

The present invention relates to methods and compositions for stabilizing a change in corneal curvature, stabilizing refractive error improvements and stabilizing (maintaining) unaided visual acuity improvements after corneal reshaping procedures. This stabilization process is accomplished by administering compositions containing at least one stabilizing ophthalmic agent to the cornea of the eye after a corneal reshaping procedure.

This patent application is based on U.S. Ser. No. 60/539,461, filed Jan. 26, 2004, the contents of which are hereby incorporated by reference, in their entirety, into the present patent application and from which priority is hereby claimed.

Throughout this application various publications are referenced. The disclosures of these publications in their entireties are hereby incorporated by reference into this application in order to more fully describe the state of the art to which this invention pertains.

### FIELD OF THE INVENTION

The present invention relates generally to the field of vision stabilization. In particular, the invention relates to methods and compositions for stabilizing corneal curvature, refractive error, and unaided visual acuity improvement in an eye. Myopia and astigmatism are stabilized after non-surgical and surgical corneal reshaping.

### BACKGROUND OF THE INVENTION

The cornea of the mammalian eye has two primary functions: protective and visual. The integrity of the cornea acts as a physical barrier for preventing the entry of noxious compounds into the underlying ocular tissues. Additionally, normal vision depends on the ability of the cornea to remain transparent and, together with the lens, transmit and refract light onto the retina. These ocular and protective functions are dependent upon the physical and chemical structure of the corneal epithelial layer, including the three-dimensional topography.

The human cornea is approximately 0.5 mm thick, and is made up of a multi-layered (i.e. stratified) epithelium with an underlying stroma. The epithelium is comprised of five to seven cell layers of epithelial cells, including three to four outer layers of flattened cells, one to three layers of mid-epithelial cells, (termed wing cells) and a single layer of column-shaped basal cells. This basal layer is the site of epithelial cell renewal through cell proliferation (i.e. mitosis) and regenerates all other epithelial layers found above. Like other stratified epithelial tissues in the human body, the cornea is self-renewing, with regeneration occurring from the basal layer every 5-7 days, resulting in a complete replacement of all epithelial cells.

The cornea is extraordinarily regular in its cellular arrangement and thickness, measuring approximately 1 mm thick peripherally, and 0.5 mm thick centrally. Because of its barrier function, the epithelial cells comprising each corneal layer are tightly associated with one another through the action of cell to cell “adherent junctions,” and their interaction with the surrounding extracellular matrix. This three-dimensional interaction and arrangement creates an epithelial tissue that acts uniformly as one unit. Specifically, it dramatically affects how the corneal epithelial tissue as a whole undergoes migration.

The capacity of corneal epithelial cells to alter their topography occurs through migration. This migratory capacity results from signaling by external cues and depends on several factors, including the internal structure of the cell and its external adhesion. The cells of the basal layer adhere tightly to an underlying substrate, called a basement membrane, through a complex adhesion system. Protein components of this basement membrane include multiple collagens, laminin and fibronectin. Interactions between the epithelial cells of the cornea and the basement membrane are critical in mediating the migration and arrangement of the individual cells, resulting in a defined three-dimensional corneal topography that determines visual acuity. In addition to the basement membrane, the epithelial and stromal cells of the cornea associate with a surrounding extracellular matrix (ECM), containing multiple collagens (types I, III, IV, V, VI, VIII, XII, XIII, XVII), proteoglycans (lumican, keratocan, mimocan), laminin and entactin. This ECM plays a critical role in cell support by providing an appropriate cellular environment and also participates in cell migration. Finally the epithelial cells of the cornea contain a well-organized internal skeleton, a cytoskeleton of filamentous proteins (actin, keratin) and microtubules, that maintain a defined cell shape, and participate in other cell functions, such as proliferation, differentiation and migration.

Approximately 80% of the refractive power of the eye is at the cornea. When the cornea is misshaped, or the axial length of the eye is too long or short, refractive errors of myopia, hyperopia and astigmatism exist. The somewhat pliable cornea can be easily reshaped to reduce and render proper spherical shape to the cornea. This correction of the corneal curvature, i.e. correction of the corneal topography, alters the cornea's light bending abilities, and improves the eye's refractive errors, leading to reduction or elimination of myopia and astigmatism and dramatically improves unaided visual acuity.

The shape of the cornea can be altered, non-surgically or surgically, to correct its shape. Non-surgical techniques such as Orthokeratology and Corneal Refractive Therapy (CRT), through the application of rigid gas permeable contact lenses, provide a temporary change in cornea shape, leading to a temporary improvement in visual acuity and refractive error. The lenses externally “re-shape” the cornea by physically inducing the migration of epithelial cells away from the central cornea without damaging the cornea. The corneal curvature is reduced in the central region and steepened in the paracentral region, resulting in a temporary improvement in the refractive errors of myopia and astigmatism and a dramatic improvement in unaided visual acuity.

However, significant regression of the corneal topography may occur after a corneal reshaping procedure. For example, studies indicate that after contact lens reshaping with Orthokeratology therapy, regression of corneal curvature, myopia and unaided visual acuity usually begins in 3-12 hours, as shown in clinical studies from the International Orthokeratology section of the National Eye Research Foundation.

Therefore, after contact lens therapy e.g., Orthokeratology with Ortho-k lenses, or CRT with rigid gas permeable CRT lenses, after maximum desired results are achieved, a retainer contact lens must be worn each night or during the day, to retain the change in corneal curvature, in order to maintain the improvements in refractive error and unaided visual acuity (UVA).

If the retainer lens is discontinued for more than 12-24 hours, significant regression occurs in the corneal topography. For example, if the baseline myopia was −3 diopters (D), and the unaided visual acuity was 20/300, and after treatment, myopia was corrected to −0.50D and 20/20 WVA, if the retainer is worn each night or day, a 20/20-20/30 WVA, and less than −1 D myopia, can be maintained. If the patient does not wear the retainer lens for a minimum of 2 days and 2 nights, the UVA may regress to 20/100 -20/200, and the myopia may regress up to −2 D to 3 D.

This regression in corneal topography to the original shape prior to the corneal reshaping procedure, is due to the corneal epithelial cells having a “memory”, as well as a definite character and shape. This “memory” ensures a predetermined epithelial cell thickness in specific regions of the cornea, leading to a specific and unique corneal shape. The epithelium reacts quickly to the application of an external force supplied by a hard, corrective contact lens (i.e. Ortho-k lenses), resulting in epithelial cell migration with no damage or detrimental effects to the cornea. However, with no retainer lens worn, there is no force holding or retaining the epithelial cells in their new position, and the corneal epithelial layer reverts back to its original topography (corneal regression). With myopia corneal reshaping, the central corneal curvature is reduced (i.e. flattened) and the paracentral curvature is steepened. With regression, the epithelial cells migrate back to the flatter central region causing steeper curvature and regression of myopia and unaided visual acuity (NBECC Clinic 1999-2003).

The corneal regressive response is, in essence, a modified wound healing response, in which the epithelial cells migrate back to their original position, thus returning visual acuity to original levels. The wound healing capacity of the cornea manifests itself in enhanced migration rates and increases in epithelial regeneration through cell proliferation from the basal layer. Therefore, the rapid migration rates of corneal epithelial cells enable practitioners to easily manipulate the shape of the cornea, and hence improve visual acuity. However, corneal regression through epithelial cell migration, along with the corneal memory, may make the correction of visual acuity using non-surgical procedures a temporary solution.

When inducing the desired change in the refractive power of the eye, the reaction of the corneal tissue is similar to that of a traumatic reaction or wound healing process observed by the compression of the central cornea and relieving the peripheral corneal area. The wound healing mechanism is the process by which migration or shifting of epithelial cells occurs during regression after corneal reshaping therapy. There is no wound after non-surgical corneal reshaping. The mechanism for wound healing stimulates epithelial cell migration during corneal curvature regression.

Regression and the stability of the visual performance after corneal refractive procedures is a major concern to patients undergoing corneal curvature correction procedures. Regression of the corneal curvature can progress to the level where the patient cannot safely perform daily distance activities such as driving, etc. without corrective lenses. Thus, a need remains for therapies to stabilize the corneal curvature after reshaping therapies.

### SUMMARY OF INVENTION

The present invention relates to compositions and methods for stabilizing changes in corneal curvature in an eye by administering to a subject a composition containing an effective amount of at least one stabilizing ophthalmic agent, after corneal reshaping procedures. The compositions are designed to retard or halt the rate of corneal epithelial cell migration, which otherwise causes regression of the shape of the altered corneal curvature.

The invention also provides methods to maintain improved unaided visual acuity and/or maintain improved refractive error by stabilizing a change in corneal curvature of the eye after a corneal reshaping procedure.

The invention also provides compositions containing stabilizing ophthalmic agents to maintain corneal curvature after corneal reshaping procedures. Vision improvements to myopia, astigmatism and unaided visual acuity, as a result of corneal reshaping, may now be maintained (stabilized) with the compositions of the invention.

Kits comprising ophthalmic agents for stabilizing corneal curvature after reshaping therapy are also encompassed by the invention. In one embodiment, a kit comprising compositions of the invention containing one or more stabilizing ophthalmic agents are used to treat an eye after a corneal reshaping procedure.

### BRIEF DESCRIPTION OF THE FIGURES

FIG. 1 is a corneal topography of myopic and astigmatic cornea.

FIG. 2 is a Post Ortho-k reshaped cornea

FIGS. 3A-C is a tissue migration picture.

FIGS. 4A-D shows corneal topography regression over-time after contact lens removal.

FIG. 5 is a Baseline Corneal topography of a myopic and stigmatic patient as described in Example 3.

FIG. 6 is a topograph showing a post Ortho-K reshaped cornea as described in Example 3.

FIG. 7 is a topograph showing corneal stabilization and regression after Ortho-K using 3 Permasight® Ophthalmic Agents in a patient as described in Example 3.

FIG. 8 is a topograph showing corneal stabilization and regression after Ortho-K using 3 Permasight® Ophthalmic Agents as described in Example 3.

FIG. 9 is a topograph showing corneal stabilization and regression after Ortho-K using 3 Permasight® Ophthalmic Agents as described in Example 3.

FIG. 10 is a topograph showing corneal stabilization and regression after Ortho-K using 3 Permasight® Ophthalmic Agents as described in Example 3.

FIG. 11 is a topograph showing corneal stabilization and regression after Ortho-K using 3 Permasight® Ophthalmic Agents as described in Example 3.

FIG. 12 is a topograph showing corneal stabilization and regression after Ortho-K using 3 Permasight® Ophthalmic Agents as described in Example 3.

FIG. 13 is a topograph showing corneal stabilization and regression after Ortho-K using 3 Permasight® Ophthalmic Agents as described in Example 3.

FIG. 14 is a topograph showing corneal stabilization and regression after Ortho-K using 3 Permasight® Ophthalmic Agents as described in Example 3.

FIG. 15 is a topograph showing corneal stabilization and regression after Ortho-K using 3 Permasight® Ophthalmic Agents as described in Example 3.

FIG. 16 is a topograph showing corneal stabilization and regression after Ortho-K using 3 Permasight® Ophthalmic Agents as described in Example 3.

FIG. 17 is a topograph showing corneal stabilization and regression after Ortho-K using 3 Permasight® Ophthalmic Agents as described in Example 3.

FIG. 18 shows corneal stabilization and regression after Ortho-K using 3 Permasight® Ophthalmic Agents as described in Example 3.

FIG. 19 is a topograph showing corneal stabilization and regression after Ortho-K using 3 Permasight® Ophthalmic Agents as described in Example 3.

FIG. 20 is a topograph showing corneal stabilization and regression after Ortho-K using 3 Permasight® Ophthalmic Agents as described in Example 3.

FIG. 21 is a topograph showing corneal stabilization and regression after Ortho-K using 3 Permasight® Ophthalmic Agents as described in Example 3.

FIG. 22 is a topograph showing corneal regression after day wear Ortho-K with no stabilization agents used as described in Example 3.

FIG. 23 is a topograph showing corneal regression after day wear Ortho-K with no stabilization agents used as described in Example 3.

FIG. 24 is a topograph showing corneal regression after day wear Ortho-K with no stabilization agents used as described in Example 3.

FIG. 25 is a topograph showing corneal regression after day wear Ortho-K with no stabilization agents used as described in Example 3.

FIG. 26 is a topograph showing corneal regression after night wear Ortho-K with no stabilization agents used as described in Example 3.

FIG. 27 is a topograph showing corneal regression after night wear Ortho-K with no stabilization agents used as described in Example 3.

FIG. 28 is a topograph showing corneal regression after night wear Ortho-K with no stabilization agents used as described in Example 3.

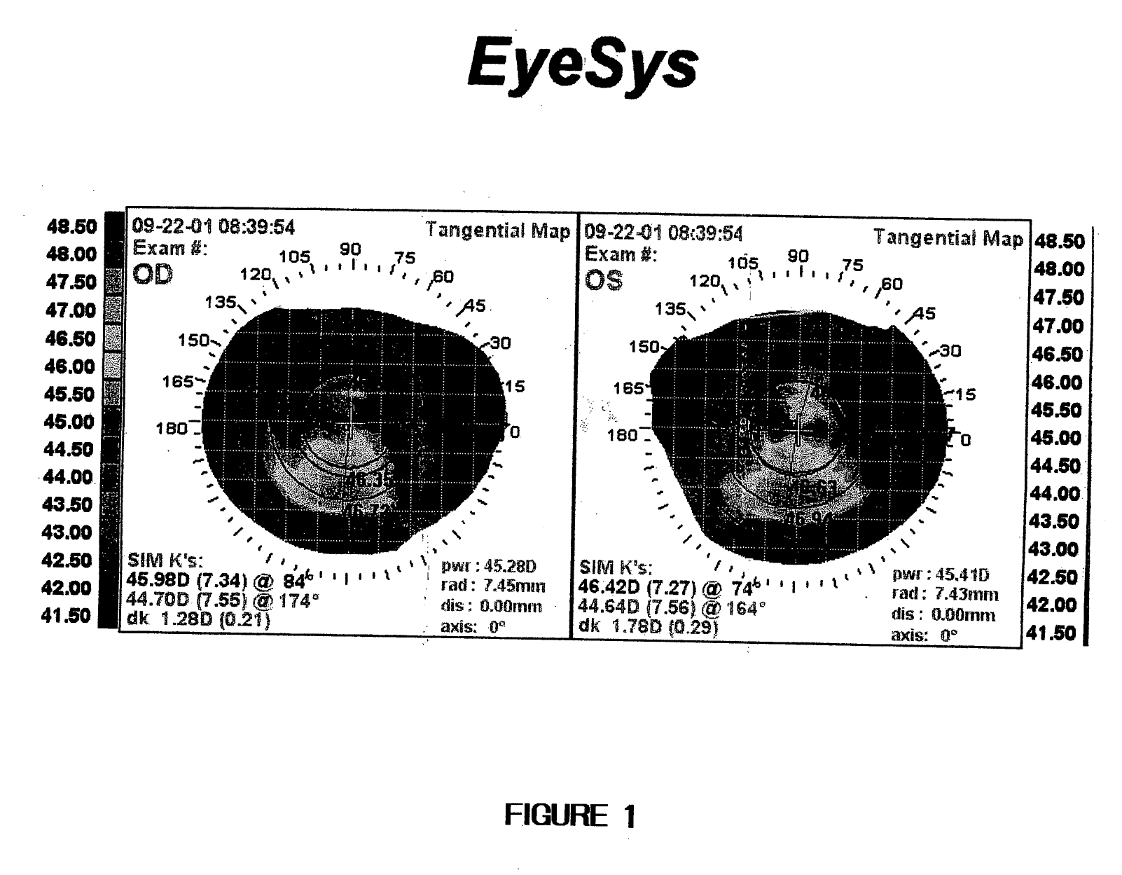

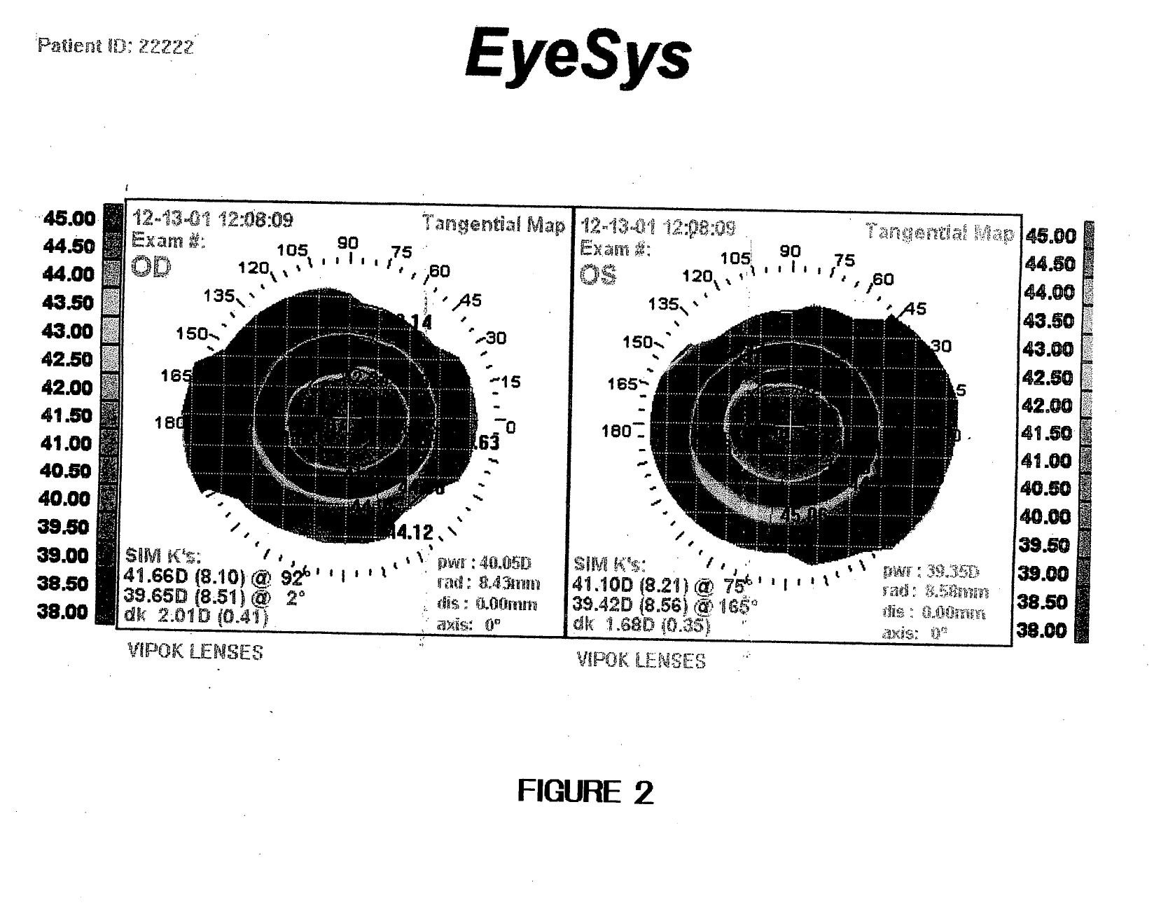

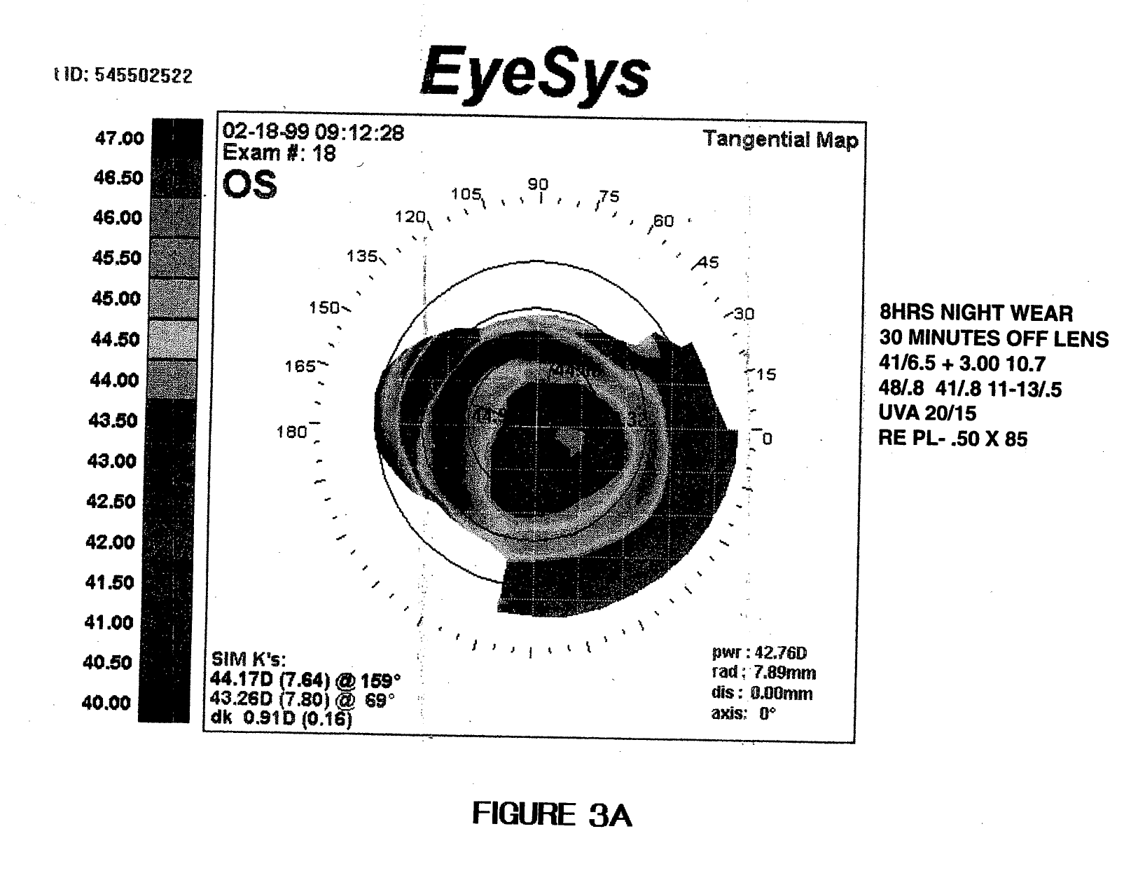

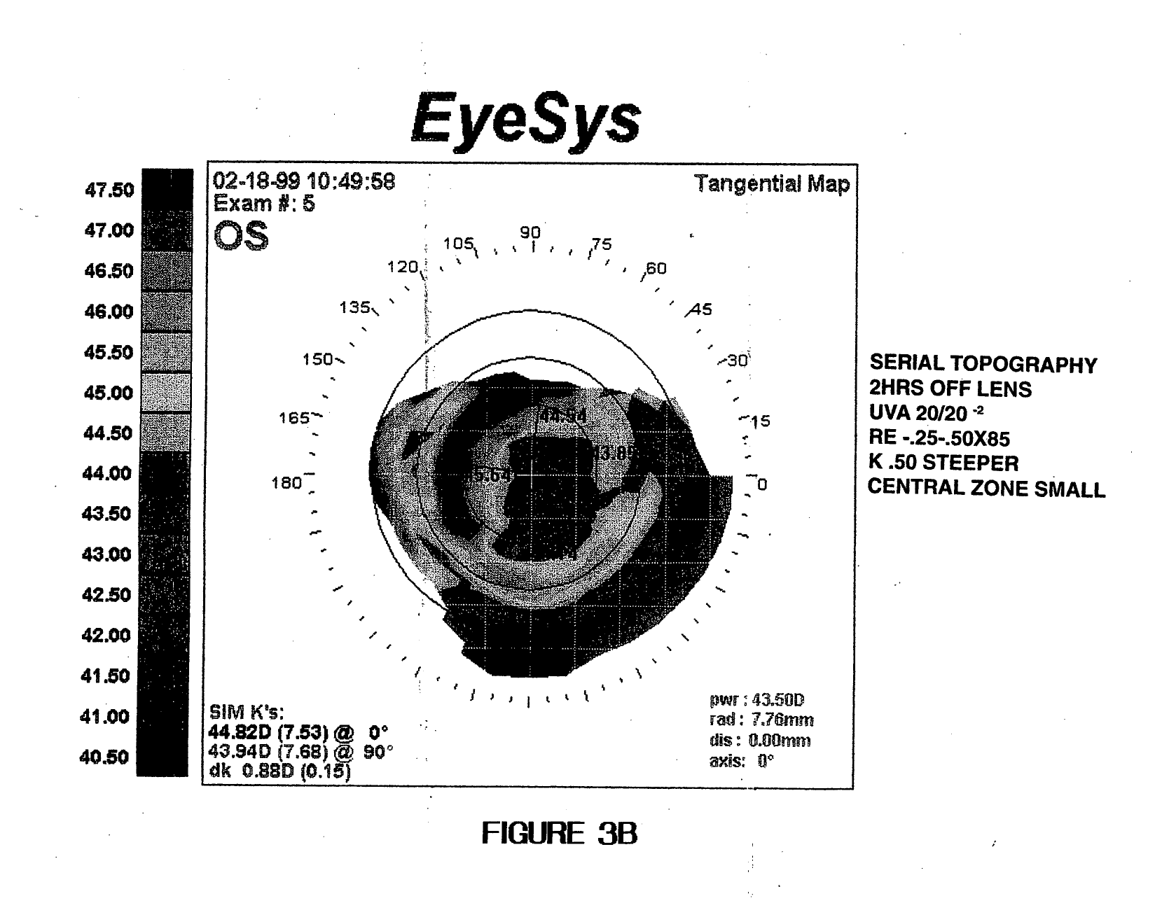

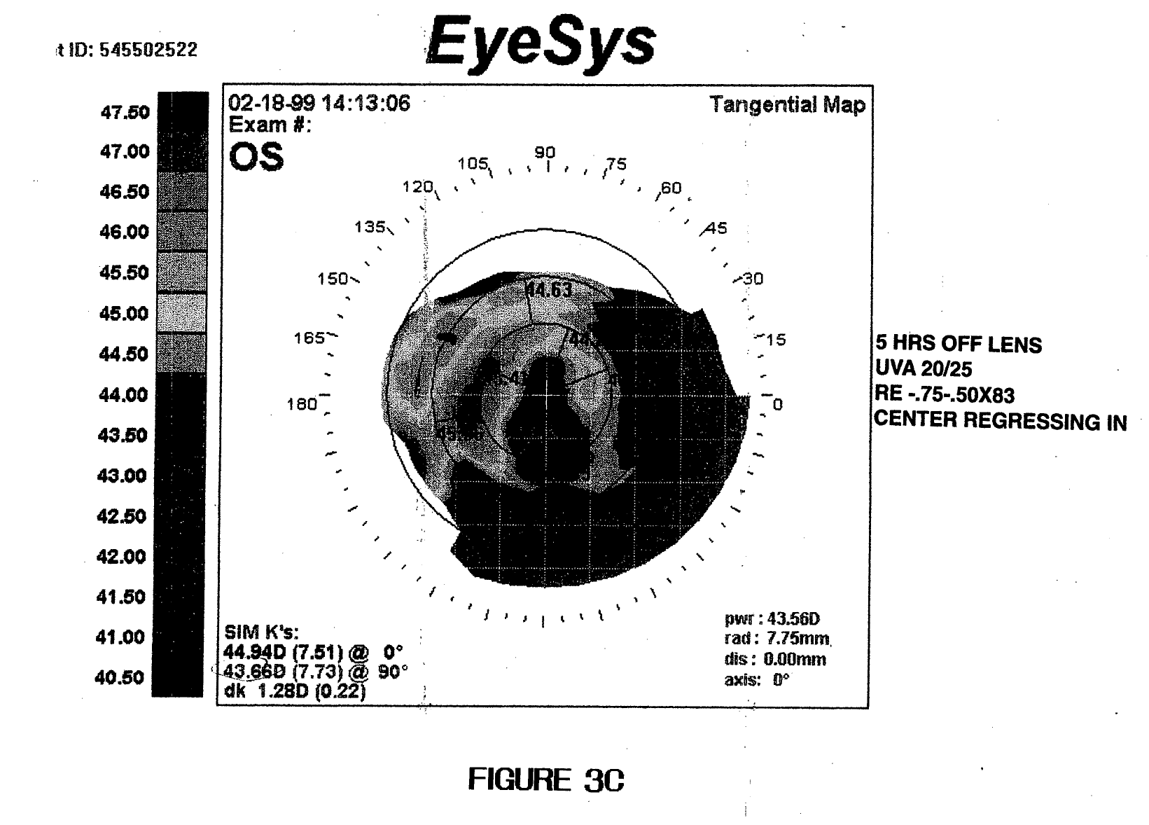

### DETAILED DESCRIPTION OF THE INVENTION

### DEFINITIONS

All scientific and technical terms used in this application have meanings commonly used in the art unless otherwise specified. As used in this application, the following words or phrases have the meanings specified.

As used herein, NSAID refers to a Non-Steroidal Anti-Inflammatory Drug. NSAIDs reduce inflammatory reactions in a subject. NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac (e.g., Acular® ketorolac tromethamine solution 0.5%), meclofenamate, naproxen, nabumetone, phenylbutazone, piroxicam, sulindac, tolmetin, acetaminophen, ibuprofen, Cox-2 inhibitors (e.g., celecoxib (Celebrex®), rofecoxib (Vioxx®) and valdecoxib (Bextra®)), meloxicam and tramadol.

As used herein, to “maintain” or “stabilize” means to render something more resistant to change or to retard or halt the effects of a process. For example to maintain or stabilize cornea curvature or topography means to render the cornea resistant to a change in its curvature, including resistance of an altered corneal curvature to regress to its original shape.

As used herein, to “reshape” or “contour” a cornea means to change the curvature of the corneal topography by any means. For example, several therapies or procedures that can reshape or contour the curvature of a cornea include, but are not limited to, non-surgical procedures such as Orthokeratology, CRT or refractive surgical procedures such as LASIK®. Reshaping or contouring a cornea can improve visual (refractive) errors of the eye such as myopia and astigmatism and improve the unaided (natural) visual acuity.

As used herein, “corneal curvature” refers to a radius of curvature measured at the center (3 millimeter) of the cornea with a Keratometer.

As used herein, “corneal topography” refers to a description, representation or feature of the shape of the cornea, i.e. a detailed number of curvature measurements of the whole cornea displayed in a computer generated color image. Specifically, a cornea is usually a positive aspheric shape (i.e. steeper in the center and flatter in the periphery. Corneal topography and axial length both contribute to common refractive errors of myopia, hyperopia and astigmatism.

As used herein, “regression” refers to a return of the corneal shape towards its original shape which can lead to increased myopia, astigmatism, and hyperopia and a loss of unaided visual acuity. The corneal topography regresses to its original shape if no maintenance or stabilizing therapy (e.g. retainer contact lenses or the ophthalmic compositions of the invention) is administered to the cornea after a corneal reshaping procedure.

As used herein, an “effective amount” is defined as an amount that can maintain or stabilize an altered corneal curvature or altered topography after cornea reshaping therapy.

As used herein, “treating” means to manage a condition by using compositions containing stabilizing ophthalmic agents, or other therapies such as surgery or contact lens correction. Treatment of an eye condition may improve the symptoms of the condition, reduce the severity of a condition, alter the course of condition's progression and/or improve the basic condition. For example, treating a myopic condition may be accomplished by regulating the curvature of the central cornea e.g., by reshaping the cornea to a new and improved shape, and maintaining the new corneal contour by administering the ophthalmic compositions of the invention.

In order that the invention herein described may be more fully understood the following description is set forth.

### COMPOSITIONS AND METHODS OF THE INVENTION

The present invention provides compositions and methods for maintaining an altered corneal curvature by administering to a subject an effective amount of an ophthalmic agent or agents after a corneal reshaping procedure. This stabilization of the corneal curvature also maintains improved unaided visual acuity and improved refractive errors of myopia, astigmatism and hyperopia.

Compositions

The present invention provides compositions for treating myopia and astigmatism by stabilizing the curvature of a cornea after the cornea undergoes a reshaping procedure.

In one embodiment, the composition of the invention contains at least one ophthalmic agent that can be administered to a subject, for example topically to the front surface or cornea of the subject's eye. The ophthalmic agent is administered to the eye of a subject after non-surgical corneal reshaping or surgical corneal reshaping, to prevent regression of corneal topography, refractive error and visual acuity.

The compositions of the invention are designed to retard or slow down the rate of corneal epithelial cell re-migration to the treated area. This prevents corneal curvature regression and vision loss. The improved results of corneal reshaping will be maintained over a much longer time period without wearing retainer contact lenses or glasses.

The ophthalmic agents used, alone or in combination, are selected based on their ability to slow down wound healing and corneal epithelial cell migration. This retards or slows down the regression of visual performance.

In an embodiment of the invention, compositions containing ophthalmic agents stabilize corneal curvature of an eye after corneal reshaping therapy by: 1) retarding or preventing epithelial cell remigration after corneal reshaping e.g. by suppressing the migration of epithelial cells to the flatter central optical zone of the cornea in myopic corneas; 2) halting or retarding the regression of the corneal curvature, refractive error and unaided visual acuity after corneal reshaping.

In another embodiment of the invention, the compositions of the invention containing ophthalmic agents that inhibit fibroblast activity from a wound healing response, leading to less scarring and/or reducing capillary permeability and cellular exudation after corneal reshaping with refractive surgery.

The present invention provides compositions containing one or more ophthalmic agent(s) that stabilize the degree of myopia and astigmatism after a corneal curvature reshaping procedure.

Examples of ophthalmic agents that can stabilize corneal curvature include, but are not limited to, agents that slow down wound healing, agents that ameliorate an immune system response, agents that change the elasticity of the cornea, agents that change the rate of epithelial cell migration, antiproliferative agents and/or agents that change the distribution of cornea cells. Ophthalmic agents include one or more of the following compounds: non steroidal anti-inflammatory agents (NSAIDs), corticosteroids, antihistamines, antibiotics mast cell stabilizers and/or metalloproteinases. Ophthalmic agents can also include tear repair agents (e.g., Restasis®), ocular anaesthetic agents (e.g., proparacaine), preservatives (e.g., benzalkonium chloride or thimerosol) and/or anti-edema agents (e.g., Muro 128®).

Compositions containing NSAIDs can provide a non-cytotoxic method of decreasing epithelial migration. In a specific embodiment, the NSAIDS include, but are not limited to, the propionic acids (including, but not limited to: fenoprofen, flurbiprofen, ketoprofen, suprofen, ibuprofen and naproxen), the acetic acids (including, but not limited to: diclofenac, etodolac, indomethacin, sulindac, tolmetin and ketorolac (e.g., Acular® ketorolac tromethamine solution 0.5%), the salicylic acids (including, but not limited to: acetyl salicylic acid, choline magnesium salicylate, magnesium salicylate, salsalate, sodium salicylate and diflunisal), the oxicams (including, but not limited to: isoxicam, piroxicam, tenoxicam and meloxicam), the fenamic acids (including, but not limited to: meclofenamate), the pyrazolones (including, but not limited to: phenylbutazone and oxyphenbutazone), the non acidic compounds (including, but not limited to: nabumetone) and the Cox-2 inhibitors (including, but not limited to: celecoxib, valdecoxib, rofecoxib). Examples of ophthalmic agents comprising NSAIDs include, but are not limited to Acular® (Ketorolac tromethamine solution 0.5%, Allergan, Inc., Irvine, Calif. 92612) and Voltaren®, (Novartis Ophthalmics, Duluth Ga. 30097).

NSAIDs are commonly used to treat ocular inflammation, and ocular itching from seasonal allergies. However, most NSAIDs also slow down wound healing and epithelial cell migration, thus affecting corneal epithelialization and reshaping. Indomethacin (an NSAID that inhibits of prostaglandin synthesis) significantly slows corneal epithelial migration below control levels. Diclofenac decreases the wound healing response of the cornea, thus increasing corneal haze. NSAIDs have also been observed to decrease gastric epithelial migration rates and slow gastric ulcer healing by interfering with growth factor actions, decreasing cell proliferation, and by altering the underlying cytoskeletal structure of the epithelial cell, thus affecting its ability to migrate. Similar effects have been observed on intestinal epithelial cells. Indomethacin, a COX inhibitor like diclofenac, results in decreased migration rates comparable to conventional NSAIDs in many epithelial tissues, including respiratory and intestinal tissues Thus, the use of NSAIDs in the compositions of the invention, can provide a non-cytotoxic method for retarding cell migration and regression after corneal reshaping.

In one embodiment of a composition of the invention, an effective amount of an ophthalmic agent such as Acular® (Ketorolac tromethamine solution 0.5%, Allergan, Inc., Irvine, Calif. 92612) is used as one drop (about 0.05 ml containing 0.25 mg), three to four times per day for one to two weeks. In another embodiment, an effective amount of Voltaren® is used as one drop, three to four times per day for one to two weeks.

In an embodiment of the invention, the compositions of the invention include a corticosteroid as the ophthalmic agent. Corticosteroids are commonly used in treatment of ocular allergies and as anti-inflammatory agents. Corticosteroids can significantly decrease migration of the corneal epithelium (for example, dexamethasone in combination with diclofenac has been shown to decrease epithelial wound healing rates following photorefractive keratectomy (PRK)), and can also inhibit keratinocyte movement leading to development of an a cellular subepithelial region following PRK ablation. However, these compounds can have side effects such as intra-ocular pressure (IOP) increase, and formation of subcapsular cataracts. Corticosteroids are commonly used in treatment of ocular allergies and as an anti-inflammatory agent. Corticosteroids such as FML® (fluorometholone 0.1%, Allergan, Inc., Irvine, Calif. 92612), prednisolone, Tobradex® (tobramycin and dexamethasone ophthalmic suspension, Alcon Laboratories, Inc. Fort Worth, Tex., 76134), Lotemax® (loteprednol etabonate 0.5%, Bausch and Lomb Pharmaceuticals, Inc., Tampa, Fla. 33637) or Alrex® (loteprednol etabonate 0.2%, Bausch and Lomb Pharmaceuticals, Inc., Tampa, Fla. 33637) are site-specific steroids that reside on the target long enough to be therapeutically effective, but rarely long enough to cause a secondary increase in IOP, and no formation of subcapsular cataracts. Loteprednol etabonate 0.2% is considered to be one of the safest of ophthalmic steroids and may be used with children.

In a preferred embodiment of a composition of the invention, an effective amount of an ophthalmic agent such as Alrex® (loteprednol etabonate 0.2%) or Lotemax (loteprednol etabonate 0.5%) is used as one drop in each eye of the subject, every six hours or two to three times a day for one week. The administration of the drops is gradually reduced to once or twice a day for a second week. In another preferred embodiment, an effective amount of FML® (fluorometholone 0.1%) is used as one drop, three to four times daily, for one week, then the number of drops is reduced gradually in a second week.

In an embodiment of the invention, the compositions of the invention include an antibiotic as the ophthalmic agent. Topical antibiotics are the most common drugs used by eye care professionals for treating eye infections such as conjunctivitis, blepharitis and keratitis. Antibiotics such as Gentamicin or Tobramycin slow down or inhibit epithelial cell migration, and slow down wound healing. Gentamicin (2%) and amphotericin B (0.1%) both decrease the healing rate/migration rate of comeal epithelium. And in some studies, Gentamicin has been shown to inhibit healing/migration of corneal epithelium more than Tobramycin.

Antimicrobial flouroquinones such as Ocuflox® (ofloxacin 0.3%), gatifloxacin, Levofloxaxcin, Ciloxan® (ciporfloxacin) and peptides e.g., polymyxin B, colistin and fosfomycin, are further examples of antibiotics that can be used as the ophthalmic agent. Fluoroquinones have become more widely used as antimicrobial agents in the treatment of multiple ocular infections and have good activity against both gram-negative and gram-positive bacteria. Higher concentrations of fluoroquinones such as oxofloxacin and ciprofloxacin and the peptides polymyxin B and colistin inhibit corneal migration rates. In addition, ciprofloxaxcin-treated corneas after PRK surgery are more to impaired or delayed wound healing. More importantly, the level of cytotoxicity of these agents appears to be low, at a wide range of therapeutic dosages, making them excellent candidates for the control of corneal migration rates

In a preferred embodiment of a composition of the invention, an effective amount of an ophthalmic agent such as Ciloxan® or Tobramycin is three drops a day, for one to two weeks. In a preferred embodiment of the invention, an effective amount of an ophthalmic agent such as Ocuflox® is the application of one drop three times a day, for one to two weeks.

In another embodiment of the invention, the composition contains a combination of corticosteroid and an antibiotic. For example, Vasocidin® is a combination of prednisolone and sulphacedamide, and Tobradex® is a combination of tobramycin and dexamethasone. These and other combinations of steroids and antibiotics are very effective in slowing down wound healing and also protecting the cornea from infection, and may be used in the compositions of the invention. An effective amount of these combination ophthalmic agents is one drop, three times a day, for one to two weeks.

In an embodiment of the invention, the compositions of the invention contain a mast cell stabilizer and/or an anti-histamine. Class Hi antihistamines have been shown to decrease dermal wound healing rates in the skin. One example of a mast cell stabilizer is Alamast® (pemirolast potassium ophthalmic solution 0.1%, Parkedale Pharmaceutical, Inc., Rochester, Mich. 48307), that inhibits the in vivo type 1 immediate hypersensitivity reaction. Pemirolast potassium also inhibits the antigen induced release of inflammatory agents e.g., histamine, from human mast cells. Pemirolast potassium is commonly used to help ocular itching and demonstrates effectiveness in slowing down smooth muscle cell migration and wound healing. Another example of a mast cell stabilizer and/or anti-histamine is Alocril® (nedocromil sodium 2%, Allergan, Inc., Irvine, Calif. 92612) and Alomide® (lodoxamide tromethamine, 0.1%, Alcon Laboratories, Inc., Fort Worth, Tex. 76134).

In another embodiment of the invention, a composition of the invention is Patanol® (olopatidine hydrochloride 0.1%, Alcon Laboratories, Inc., Fort Worth, Tex. 76134) includes a mast cell stabilizer and an antihistamine. Olopatadine inhibits the release of 25 histamine from the mast cells and is an antagonist that inhibits type I immediate hypersensitivity reaction. It is used for the treatment of allergic conjunctivitis. In addition, this compound also suppresses the antigen-presenting ability of the murine immune system and therefore may slow down epithelial cell migration and wound healing.

In a preferred embodiment of a composition of the invention, an effective amount of an ophthalmic agent such as Alamast® is one drop, three times a day, for one to two weeks. In another preferred embodiment, an effective amount of an ophthalmic agent such as Patanol® is one drop, twice a day, for one to two weeks.

In an embodiment of the invention, the compositions of the invention include a metalloproteinase inhibitor as the ophthahnic agent. Metalloproteinases include compounds which can degrade the extracellular matrix, such as MMP-2, MMP-9. Increases in MMPs have been detected after corneal wounding and overexpression of MMPs by resident corneal cells can interfere with corneal re-epithelialization/corneal migration. Blocking gelatinase B (MMP-X) decreases human respiratory epithelium repair speed. In one example, the metalloproteinase inhibitor is Ilomastat (Arriva Pharmaceuticals, Inc., Alameda, Calif. 94501), which can inhibit cellular migration

In an embodiment of the invention, the compositions of the invention include ocular anesthetics. Ocular anesthetics such as proparacaine (proxymetacin HCl) are used to relieve ocular pain. Other anesthetics such a procaine and bupivacine can reduce ocular/corneal pain and show no significant toxicity to cell structure. A side benefit of ocular anesthetics is that they retard wound healing and epithelial cell migration. Local anesthetics such as lidocaine (0.5 to 2%) can impair corneal migration/re-epthelialization rates and proparacaine decreases the wound closure/migration rate of rabbit corneal epithelium. Ocular anesthetics such as proparacaine can be used when the patient sleeps with his/her retainer lens, to assist stability and to control irritation of the eye caused by the retainer lens.

In a preferred embodiment of a composition of the invention, an effective amount of an ophthalmic agent such as proparacaine is used as 1 drop of a 0.5% solution every 4 hours.

Preservatives such as Thimerisol and Benzalkonium chloride are commonly added to solutions containing other ophthalmic agents such as antibiotics, to preserve the shelf life of the agent, however, preservatives may also have the added benefits of retarding wound healing and cell migration. These preservatives can be added to other ophthalmic agents used to stabilize corneal curvature.

In a preferred embodiment of a composition of the invention, an effective amount of an ophthalmic agent such as Benzalkonium chloride is 0.004% to 0.01% volume weight of the solution to which the preservative is added.

In an embodiment of the invention, the compositions of the invention include agents that reduce edema. For example, Muro 128® (sodium chloride hypertonicity ophthalmic solution 5%, Bausch and Lomb Pharmaceuticals, Inc., Tampa, Fla. 33637) is an anti-edema agent that can reduce edema caused by contact lens therapy decreasing corneal hydration while reshaping the cornea. Muro 128® (& can improve unaided vision after corneal reshaping therapy and reduce edema after sleeping.

In a preferred embodiment of a composition of the invention, an effective amount of an ophthalmic agent such as Muro 128® is one drop in the morning applied to each eye after sleeping in retainer contact lenses.

In an embodiment of the invention, the compositions of the invention include other agents that have been found to inhibit or decrease epithelial migration rates. Use of many of these agents is based on their effects on the histologic structure of the cornea and the mechanisms of corneal epithelial cell adhesion. For example, platelet activating factor (PAF) remodeling of the extracellular matrix can dramatically inhibit adhesion of corneal epithelial cells, a fact that could significantly alter migration capacity. Additionally, alteration of the basement membrane of the cornea can also have dramatic effects on cell migration. For example, inhibitors of collagen and hyaluronan synthesis can disrupt the spread of corneal epithelial sheets. In another example, administration of antibodies to the corneal proteoglycan lumican can inhibit epithelial migration in organ culture systems. Corneal epithelial healing has been shown to be delayed in lumican-deficient mice. Also, administration of a compound such as polysufated glycosaminoglycans has been shown to have a concentration-dependant effect on corneal cell morphology and migration. In addition, the anti-proliferative effects of colchicine on lens epithelium may also inhibit migration. Like microtubules, the sythesis of cytoskeletal proteins such as actin, talin and vinculin, in addition to cell surface proteins such as CD44 and the hyaluronan receptor, is critical to epithelial migration. Disruption of the actin cytoskeleton by agents such as proparacaine can inhibit corneal epithelial migration.

In an embodiment of the invention, the compositions of the invention include immunosuppressive agents such as cyclosporin, azathioprine, methotrexate, TNFα blockers or antagonists, or a biological agent targeting an inflammatory cytokine, hydroxychloroquine, sulphasalazopryine, gold salts, etanercept, infliximab, rapamycin, mycophenolate mofetil, azathioprine, tacrolismus, basiliximab, cytoxan, interferon beta-1a, interferon beta-1b, glatiramer acetate, mitoxantrone hydrochloride, anakinra and/or other biologics. For example, Restasis® (cyclosporine ophthalmic emulsion 0.05%, Allergan, Inc., Irvine, Calif. 92612), which is commonly used to repair the tear layer of the eye for dry eye disease patients, also has the ability to slow down epithelial cell migration.

In a preferred embodiment of a composition of the invention, an effective amount of an ophthalmic agent such as Restasis® (cyclosporine 0.05%) is two drops a day for one to two weeks or longer.

Compositions of the invention can also include other compounds for stabilizing the cornea. These stabilizing ophthalmic agents can have one or more of the following attributes: they increase the rigidity of the cornea; they reduce or increase the pliability & elasticity of the cornea; they retard or halt epithelial cell migration, or redistribution of cells; they decrease proliferation of cells; they assist in cross-linking or bonding of corneal cells, for example, ultraviolet light; they increase epithelial glycogen; they increase A.T.P.; they help control glucose utilization; and they increase lactate dehydrogenase.

As is standard practice in the art, the compositions of the invention, comprising pharmaceutically effective amounts of the stabilizing ophthalmic agents, are admixed with an acceptable carrier or adjuvant known to those of skill of the art. The compositions of the invention are usefull for treatment of an eye after a corneal reshaping procedure, to stabilize the change in corneal curvature.

The compositions preferably include suitable carriers and adjuvants which include any material which, when combined with the stabilizing ophthalmic agents of the invention (e.g., Acular®, Patanol® or Ciloxan®), retain the ophthalmic agent's activity, and are non-reactive with a subject's immune system. These carriers and adjuvants include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, phosphate buffered saline solution, water, emulsions (e.g. oil/water emulsion), salts or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances and polyethylene glycol. Other carriers can also include sterile solutions; tablets, including coated tablets and capsules. Typically such carriers can contain excipients such as starch, milk, sugar (e.g. sucrose, glucose, maltose), certain types of clay, gelatin, stearic acid or salts thereof, magnesium or calcium stearate, talc, vegetable fats or oils, gums, glycols, or other known excipients. Such carriers can include flavor and color additives or other ingredients. Compositions comprising such carriers are formulated by well known conventional methods. Such compositions may also be formulated within various lipid compositions, such as, for example, liposomes as well as in various polymeric compositions, such as polymer microspheres. Such carriers can also include preservatives, such as Thimerisol, or Benzalkonium chloride.

Kits comprising the compositions of the invention are also useful for stabilizing reshaped corneal curvature in the methods of the invention. In one embodiment, the kit comprises a composition that includes one or more of the stabilizing ophthalmic agents, for example, Acular®, Patanol® and/or Ciloxan®, with an acceptable carrier or adjuvant, e.g., a pharmaceutically acceptable buffer, such as phosphate-buffered saline, Ringer's solution and dextrose solution. The kit may further include other materials desirable from a commercial end user standpoint, including other buffers, diluents, filters, needles, syringes, eye-drops and package inserts, with instructions for use. The compositions of the invention may be provided as a sterile solution or as dry powders, usually lyophilized, including excipients that upon dissolving will provide a reagent solution having the appropriate concentration. The components of the kit, containing one or more of the compositions of the invention, are used to stabilize the change in corneal curvature, after a corneal reshaping procedure.

The compositions of the invention, alone or in combination with other ophthalmic agents, can be used as a corneal curvature stabilizing agent. Alternatively, compositions containing one or more of the stabilizing ophthalmic agents, along with the use of retainer contact lens can be used to stabilize corneal curvature. A reverse geometry lens made of silicone/acrylate material is the most common reshaping and retainer contact lens. It is comprised of a flat central optic zone and a steep peripheral zone surrounding, an alignment curve and a peripheral curve. Retainer lenses with spherical base curves and aspheric base curves, in the same material, can also be used.

Administration of the compositions of the invention containing one or more stabilizing ophthalmic agents, alone, or in combination, to the cornea, can stabilize the corneal topography after a corneal reshaping procedure and retard the rate of corneal regression and epithelial cell migration. This stabilization of the reshaped cornea permits the eyecare professional to maintain the improved unaided visual acuity and refractive error changes from the corneal reshaping procedure, without the use of artificial aids, such as eyeglasses, or daily wear contact lenses. Stabilization of the corneal curvature allows a patient to see clearly, without glasses or contact lenses, for an extended period.

### METHODS OF THE INVENTION

The present invention also provides methods for stabilizing changes in corneal curvature after a corneal reshaping procedure. The methods comprise administering to the subject an effective amount of a composition containing at least one stabilizing ophthalmic agent, to maintain the reshaped topography of the cornea, for example after subjecting the cornea of an eye to corneal reshaping procedures to correct its refractive error and to normalize unaided visual acuity and stabilize the improved refractive error. The present invention provides methods for maintaining improved unaided visual acuity and refractive error, after a corneal reshaping procedure, by stabilizing the corrected corneal curvature.

The invention provides methods for correcting myopia and astigmatism by stabilizing corneal curvature after a corneal reshaping procedure. Myopia and astigmatism are reduced or eliminated in a subject, by reshaping and correcting the curvature of a cornea, then administering an effective amount of the composition of the invention, containing at least one ophthalmic agent(s), to maintain the reshaped curvature of the cornea.

In accordance with the practice of the invention, the methods comprise reshaping the cornea of the eye by methods well known in the art such as nonsurgical methods e.g., Orthokeratology, CRT, Corneaplasty (Orthokeratology with enzymes) or refractive surgical methods e.g., LASIK, Photorefractive Keratectomy (PRK), radial keratometry; Intracorneal Rings (ICR), or methods employing radiowaves. In one embodiment, myopic corneal reshaping reduces corneal thickness centrally, and increases corneal thickness paracentrally, i.e., reduces (flattens) the central curvature of the cornea.

In accordance with the practice of the invention, the methods comprise administering to a subject a composition of the invention containing one or more stabilizing ophthalmic agent(s), in an effective amount to stabilize corneal curvature after a reshaping procedure. The effective amount inhibits re-migration of corneal epithelial cells to the center of the cornea, i.e. inhibits regression of the epithelial cells back to the center of the cornea (steepening the central curvature), while reducing the paracentral curvature and thickness.

Administration of an ophthalmic agent(s) of the invention stabilizes improved unaided visual acuity change by: 1) retarding and/or preventing the epithelial cells from shifting back to the central cornea i.e. preventing regression of the central curvature of the cornea (i.e. central K) to a steeper curvature;, 2) halting or retarding corneal and refractive regression after surgical corneal reshaping, 3) halting or retarding corneal and refractive regression after non-surgical corneal reshaping and after removal of a therapeutic lens; 4) suppressing re-migration of epithelial cells to the flatter optical zone of the cornea; 5) suppressing re-migration of the cells to the site of the alteration in corneal curvature caused by the reshaping procedure.

In an embodiment, a composition of the invention is administered to a subject, during a corneal reshaping procedure. In another embodiment of the invention, a composition of the invention is administered to a subject, after a corneal reshaping procedure. In a further embodiment, a composition of the invention containing at least one stabilizing ophthalmic agent(s) is administered to a subject, before a corneal reshaping procedure. Alternatively, the compositions containing stabilizing ophthalmic agent(s) can be administered to a subject in any combination, before, during and/or after, a corneal reshaping procedure. The soluble compositions of the invention can be administered to a subject in an amount and for a time (e.g. length of time and/or multiple times) sufficient to maintain the corneal curvature of the eye. In addition, a retainer contact lens may be used. For example, the ophthalmic agents can be administered to a subject two to three times a day, for a minimum of two weeks. More time can be added as necessary, along with minimal retainer contact lens wear.

In accordance with the invention, administering of the compositions of the invention can comprise co-administration, e.g. concomitantly or in sequence, of a therapeutically effective amount of a composition of the invention, with one or more additional compositions of the invention. Administration can also comprise a continuous release (time-release) of the agent(s), for example, the agent(s) can be embedded in a time-release capsule or other continuous release material.

The subjects treated by the present invention include mammalian subjects, including, humans, monkeys, apes, dogs, cats, cows, horses, goats, pigs, rabbits, mice and rats.

The present invention provides various methods, local or systemic, for administering the compositions of the invention. As is standard practice in the art, the compositions of the invention may be administered to the subject in any pharmaceutically acceptable form.

The methods include iontophoresis injections, as topical eye drops, in gel form, intravenous, intramuscular, intraperitoneal, oral, inhalation and subcutaneous methods, as well as by implantable pump, continuous infusion, gene therapy, liposomes, suppositories, topical contact (for example the opthalmic agent is embedded in a contact lens, is embedded in a gel form and placed underneath the eyelid or is embedded in a drug delivery patch), vesicles, capsules, biodegradable polymers, hydrogels, controlled release patch and injection methods. The composition of the invention, compounded with a carrier, is commonly packaged as a sterile solution to be administered directly to an eye topically. In a preferred embodiment, the compositions of the invention containing the ophthalmic agent(s) are administered directly to an eye in a topical liquid solution (i.e. eye drop).

### EXAMPLE 1

### Probe Study #1 Showing Corneal Stabilization with Acular® After Non-Surgical Ortho-K Corneal Reshaping

Acular ® (ketorolac tromethamine solution 0.5%, Allergan, Inc., Irvine, Calif. 92612), a non-steroidal anti inflammatory agent, has proven to be effective in slowing down the regression and epithelial cell migration after corneal reshaping. The study described herein used Acular®, administered as a drop three times per day for one week, to stabilize corneal topography after Ortho-K therapy.

Cohort

Fifteen (15) subjects ranging in age from 21-45 years old, with myopia ranging from negative one to negative three diopters and having less than negative one diopter of astigmatism were recruited for the study. The study excluded pregnant, lactating or hormone imbalanced women.

Probe Study Procedures

The fifteen (15) subjects underwent Orthokeratology (Ortho-K) contact lens therapy. The non-dominant eye's cornea was reshaped towards less central curvature, with RGL (rigid gas permeable lenses) therapeutic contact lenses to reduce their myopia and improve their unaided visual acuity to a normal functioning level. Each subject slept in their Ortho-K night wear lenses for 6-8 hours and used lubrication drops e.g., saline drops, every morning (a.m.) and evening (p.m.).

On the first day of the study, each subject wore their lenses into their ophthamologist's office. After 1 night of corneal reshaping (average 8 hours) or 1 day of corneal reshaping (average 12 hours), the contact lenses were removed and an eye exam was performed to measure the improvements in corneal curvature, myopia, unaided visual acuity and cornea integrity as a baseline to compare corneal stabilization results. The average time off the contact lenses for the exam varied between 15 minutes and 2 hours.

After the initial eye examination, one drop of Acular® (0.5% solution with each drop was approximately 0.05 ml and contains about 0.25 mg of) was administered into the non-dominant eye (head back, eye closed) three times a day for seven days. The dominant eye was used as a control and Acular® was not administered to it.

Retainer contact lens wear was discontinued for the duration of the study. Administration of Acular® drops was discontinued after seven days of use.

Home Testing

Each subject was required to test and record their unaided visual acuity 2-3 times per day (morning and evening) for seven days using a home Ortho-K visual acuity chart in good lighting at 10 feet away.

Doctor's Office Testing

Each subject was requested to return to their ophthalmologist's office for follow-up monitoring according to the following schedule:
Day 1: am & late afternoon.
Day 2: am only.
Day 3: Late afternoon.
Day 4: No exam.
Day 5: am only.
Day 6: No exam.
Day 7: pm only.

The subjects were measured for the following categories: 1) unaided visual acuity, 2) objective and subjective refraction, 3) corneal curvature and topography, and 4) comfort and vision comments.

Results From 15 Patients

1. Unaided Visual Acuity
Average baseline U.V.A. before corneal reshaping=20/250
Average improvement in U.V.A. after 1 night (8-12 hours) of Ortho-k reshaping=9 lines of U.V.A. improvement =20/250 to 20/25
Average U.V.A. after 7 days of Acular® (3 times a day) and no contact lens wear=20/40 U.V.A. (2 lines regression). Good retention of improvements after 7 days of stabilization.

2. Refractive Error
Average baseline myopia=−2.5 Diopters (D)
Average refractive error after 8-12 hours of Ortho-k corneal reshaping=−0.50 D net change of myopia from −2.5 D to −0.50 D=a net improvement of −2.00 D
Average refractive error after 7 days of Acular® 0.5% (3 times a day) and no retainer contact lens wear=−1.50 D. Net regression=1 D myopia over 7 days of corneal stabilization.

3. Central Corneal Curvature (Flattest Meridian)
The average beginning central K before reshaping=43.87 D.
The average central corneal curvature after 8-12 hours of Ortho-k reshaping=42.50 D, net change=1.37 D flatter.
Average central corneal curvature after 7 days of Acular® 0.5% (3 times a day) and no retainer contact lens wear=43.12 D. Net regression of central K=0.62 D steeper curvature.

4. Patient Symptoms and Side Effects
Slight burning upon administration of Acular® drops (preservative in Acular®).
A temporary tight and constricted feeling in treated eye of 30% of patients.
Patients excited with vision holding without retainer lens.
Most patients could perform daily activities with no lenses (driving, sports, movies, etc).

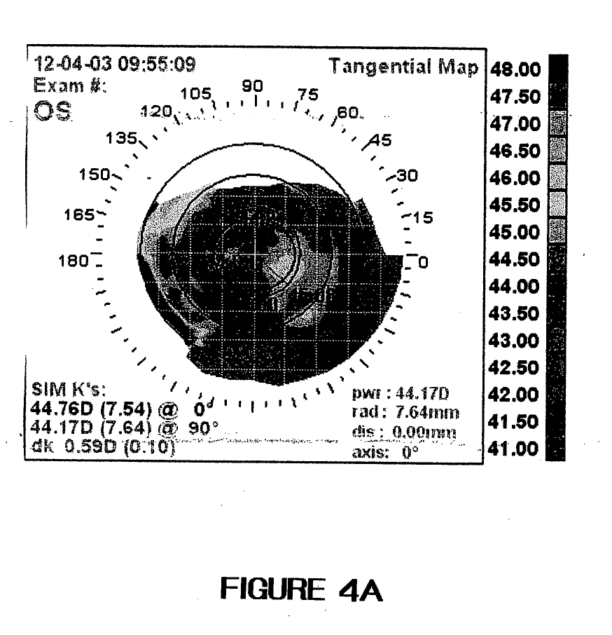

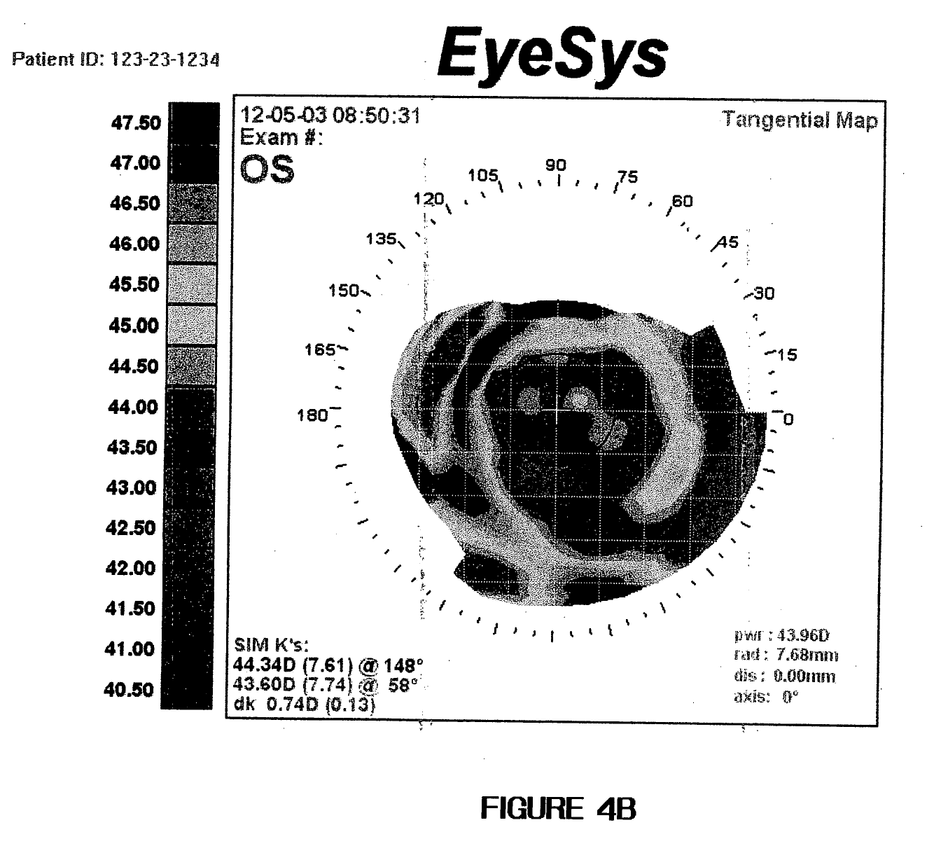

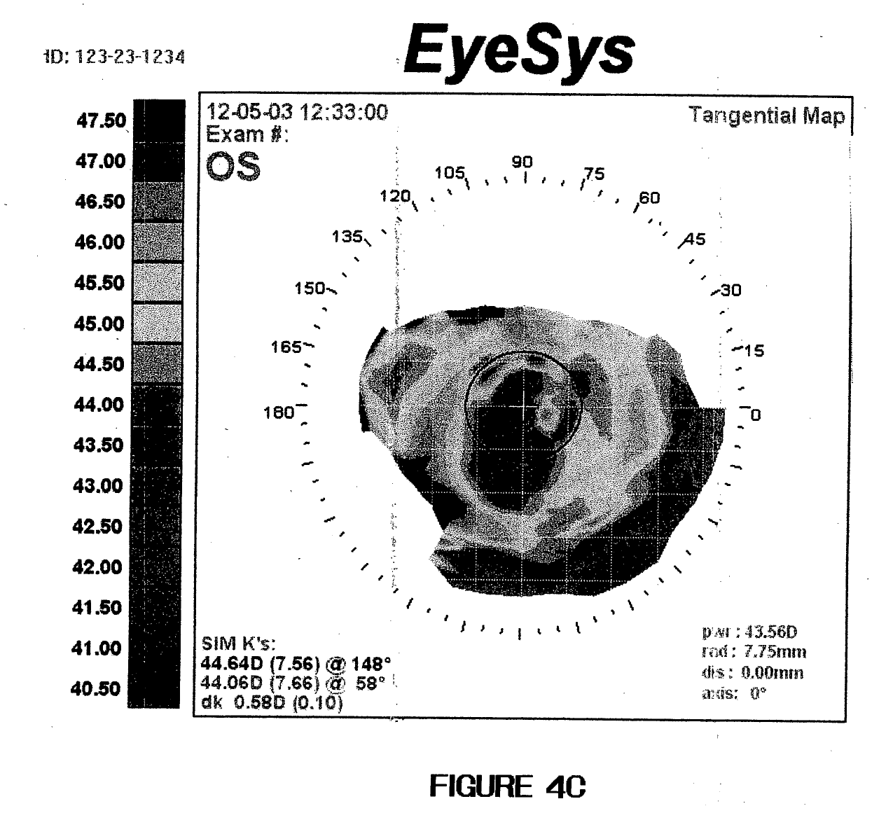

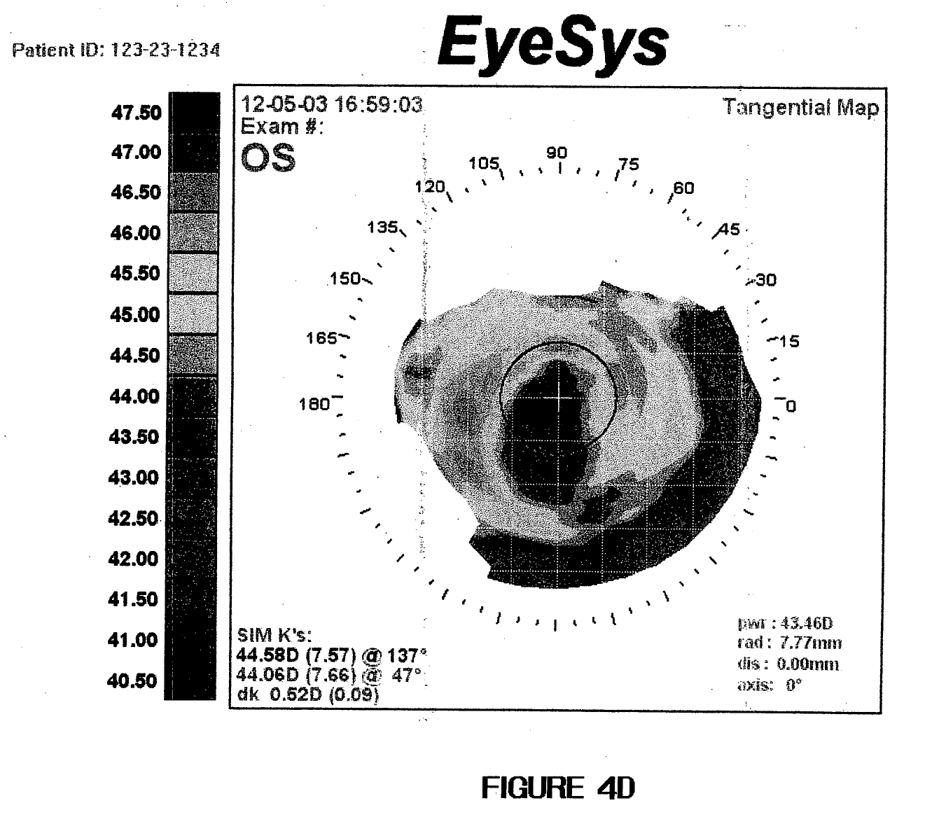

After nonsurgical ortho K corneal reshaping, most patients complain about their vision loss after removal of their retainer lens for twelve to eighteen hours. However, the subjects of the subject study were able to go without significant vision loss while using Acular® drops three times a day for seven days with no retainer lens wear. The patients exhibited little or no regression of refractive error and corneal curvature (FIG. 4). These patients were able to function normally during the day in far and near activities such as driving, athletics, computer and reading activities with no contact lens wear or glasses. Unaided visual acuity was maintained at 20/20-20/40.

This study indicates that using Acular® retarded the regression of myopia significantly by stabilizing corneal curvature after a corneal reshaping procedure.

### EXAMPLE 2

### Probe Study #2, Corneal Stabilization with Three Therapeutic Pharmaceutical Agents After Non-Surgical Ortho-K Corneal Reshaping

Therapeutic Pharmaceutical Agents (T.P.A.) in combination to be tested:

| T.P.A. | Classification | Dosage & Schedule |
|---|---|---|
| Acular ® (0.5%) | NSAID | TID (3 times a day) |
| Patanol ® (0.1%) | Antihistamine | TID (3 times a day) |
| | mast cell stabilizer | |
| Ciloxan ® (0.3%) | anti-infective antibiotic | TID (3 times a day) |

Cohort

Subjects to be recruited for this study must abide by the following criteria: subjects must range in age from 21-40 years old, with myopia less than negative three diopters and having less than negative one diopter of astigmatism were recruited for the study. The study will exclude pregnant, lactating or hormone imbalanced women.

Probe Study Procedures:
Ortho-K patients using night wear retainer contact lenses.
Sleep in Ortho-K night wear lenses 6-8 hours (lubrication gtt am/pm).
Am 1^{st} day, wear your lenses into the DR's office after sleeping.
Am—remove retainer lenses & instill study T.P.A.'s as follows:
1 drop Acular in non-dominant eye (head back, eye closed).
1 drop Patanol into same eye 5 minutes after 1^{st} drop.
1 drop Ciloxan into same eye 5 minutes after 2^{nd} drop.
At approximately 12:00 pm (noon) repeat the am regimen (with 3 agents above).
After 7:00 pm repeat the 3-drop routine same as above regimen (with 3 agents above).
No contact lens wear for 7 days.
Repeat drops with 3 agents, 3×/day for the next 6 days.
   Home Testing

Each subject will be required to test and record their unaided visual acuity 2-3 times per day for seven days using a home Ortho-K visual acuity chart in good lighting at 10 feet away.

Doctor's Office Testing

Each subject will be requested to return to their optometrist's/ophthalmologist's office for follow-up monitoring according to the following schedule:
Day 1: am & late afternoon.
Day 2: am only.
Day 3: Late afternoon.
Day 4: No exam.
Day 5: am only.
Day 6: No exam.
Day 7: pm only.

The subjects will be tested by an optometrist's/ophthalmologist for the following categories:
Comfort and vision comments.
Unaided visual acuity.
Objective and subjective refraction.
Corneal curvature and topography.
Slit lamp.

Preliminary data suggest that combinations of Acular®, Ciloxan® and Patanol® seem effective in retarded the regression of myopia by stabilizing corneal curvature after a corneal reshaping procedure.

### EXAMPLE 3

### Results of Corneal Stabilization with Three Therapeutic Pharmaceutical Agents After Non-Surgical Ortho-K Corneal Reshaping

This example provides the results from 8 subjects using 3 Permasight Ophthalmic Agents using the protocol of Example 2.

1. Unaided Visual Acuity
Average baseline U.V.A. before corneal reshaping=20/200
Average U.V.A. after Ortho-K corneal reshaping=20/30.
Average improvement in U.V.A.=7 lines.
Average U.V.A. after seven (7) days without contact lenses using three (3) different drops for three (3) times a day=20/80 U.V.A.
Net U.V.A. regression=5 lines (20/30 to 20/80)

2. Refractive Error
Average baseline myopia=−2.0 Diopters (D).
Average refractive error after 8 hours of Ortho-k corneal reshaping=−0.50 D.
Average myopia improvement=1.50 D.
Average refractive error after seven (7) days of no contact lens wear using three (3) Permasight Ophthalmic agents=−1.35 D.
Net refractive error regression=−0.85 D (−0.50 D to −1.35 D).

3. Central Corneal Curvature “K” (Flattest Meridian)
The average beginning central K before reshaping=43.00 D.
The average central corneal curvature after 8-12 hours of Ortho-k reshaping=42.00 D.
Average central corneal curvature after seven (7) days of no retainer contact lens wear and with three (3) different drops for three (3) times a day)=42.71 D.
Net corneal curvature regression=0.71 D.

These data demonstrate that combinations of Acular®, Ciloxan® and Patanol® are effective in retarding the regression of myopia and U.V.A. by stabilizing corneal curvature after a corneal reshaping procedure.

Corneal Topography Results

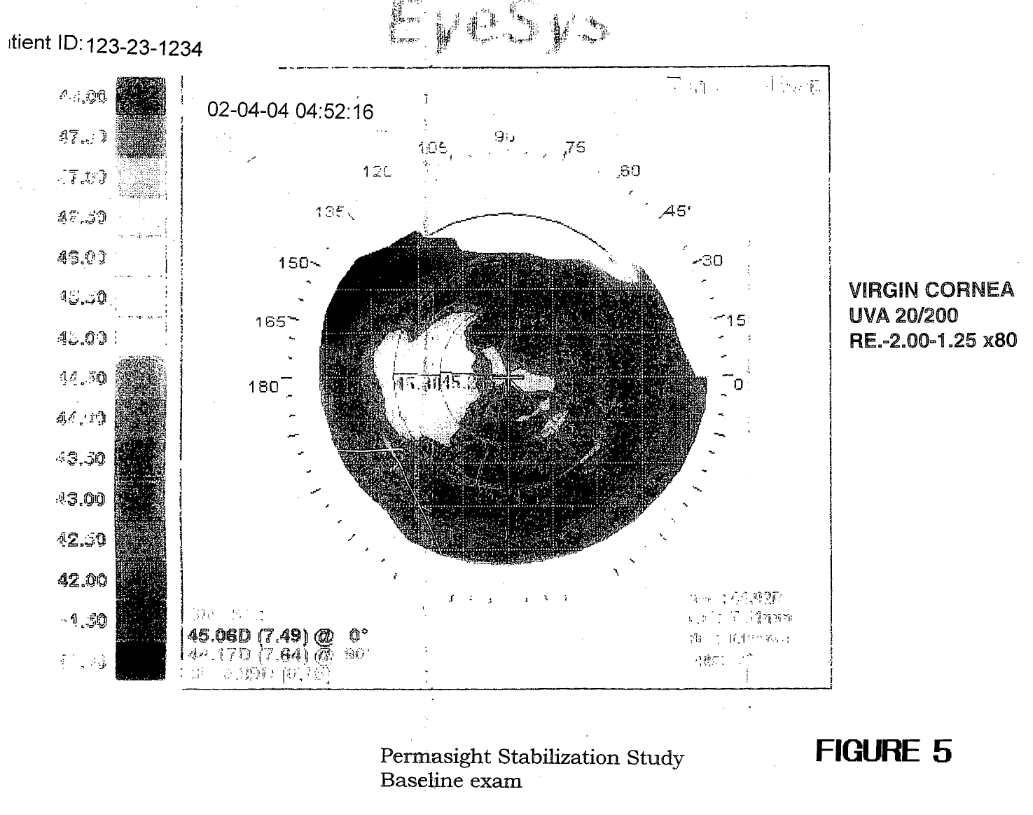

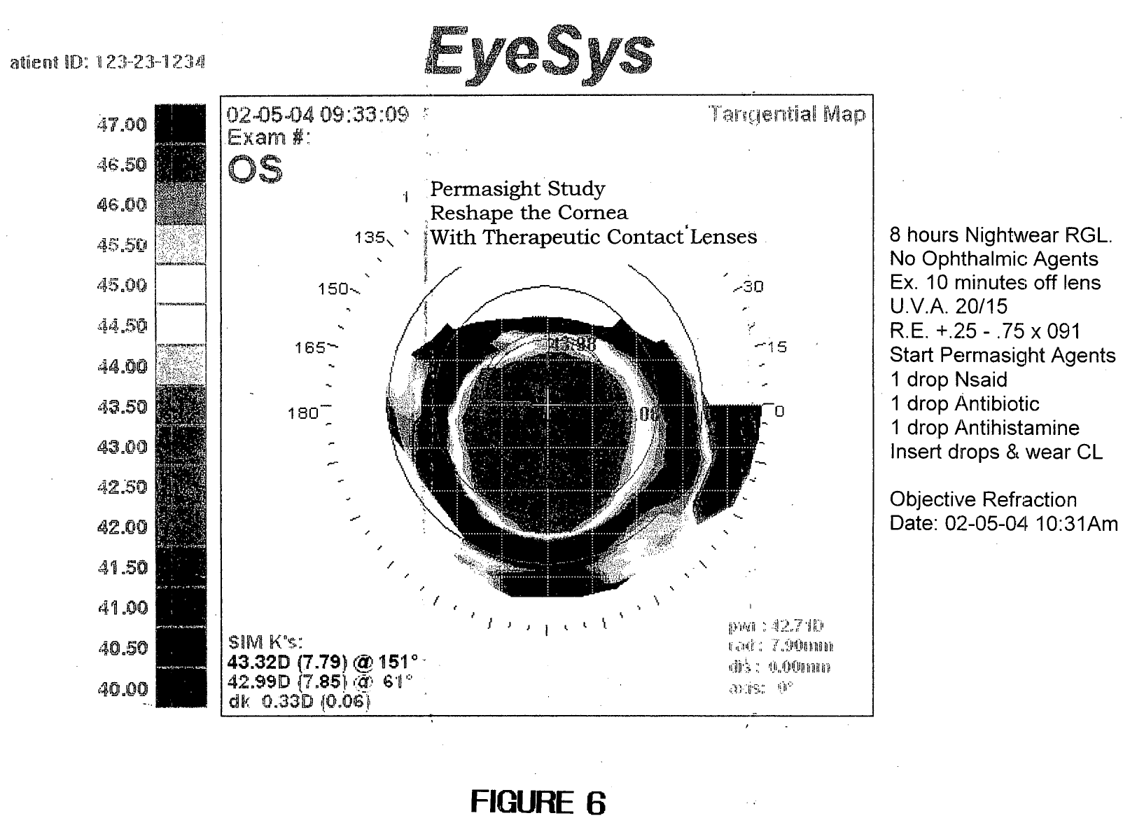

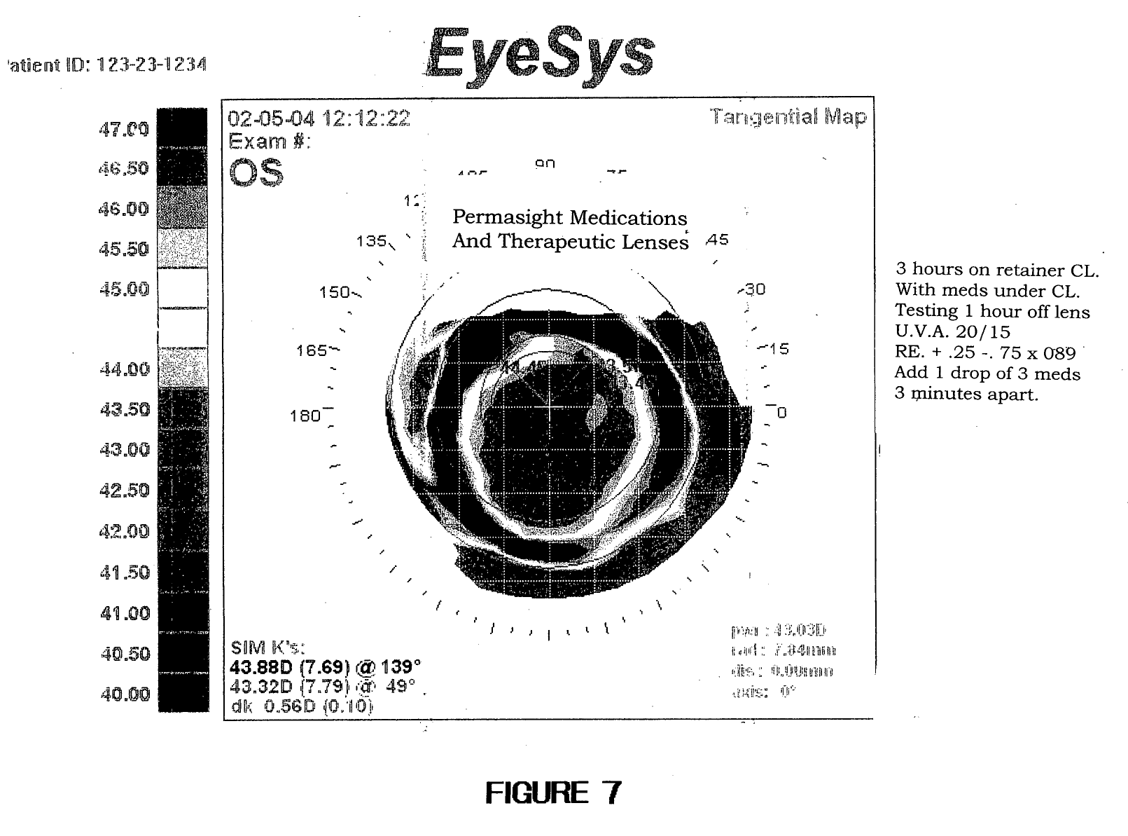

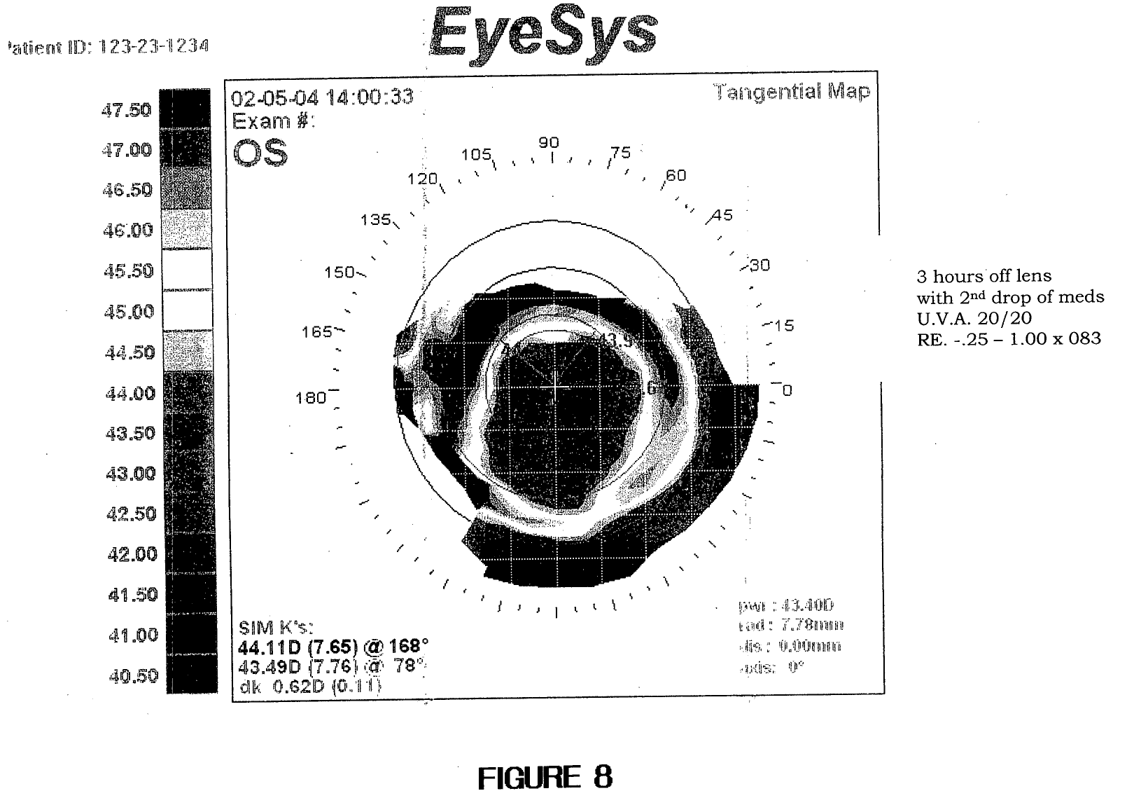

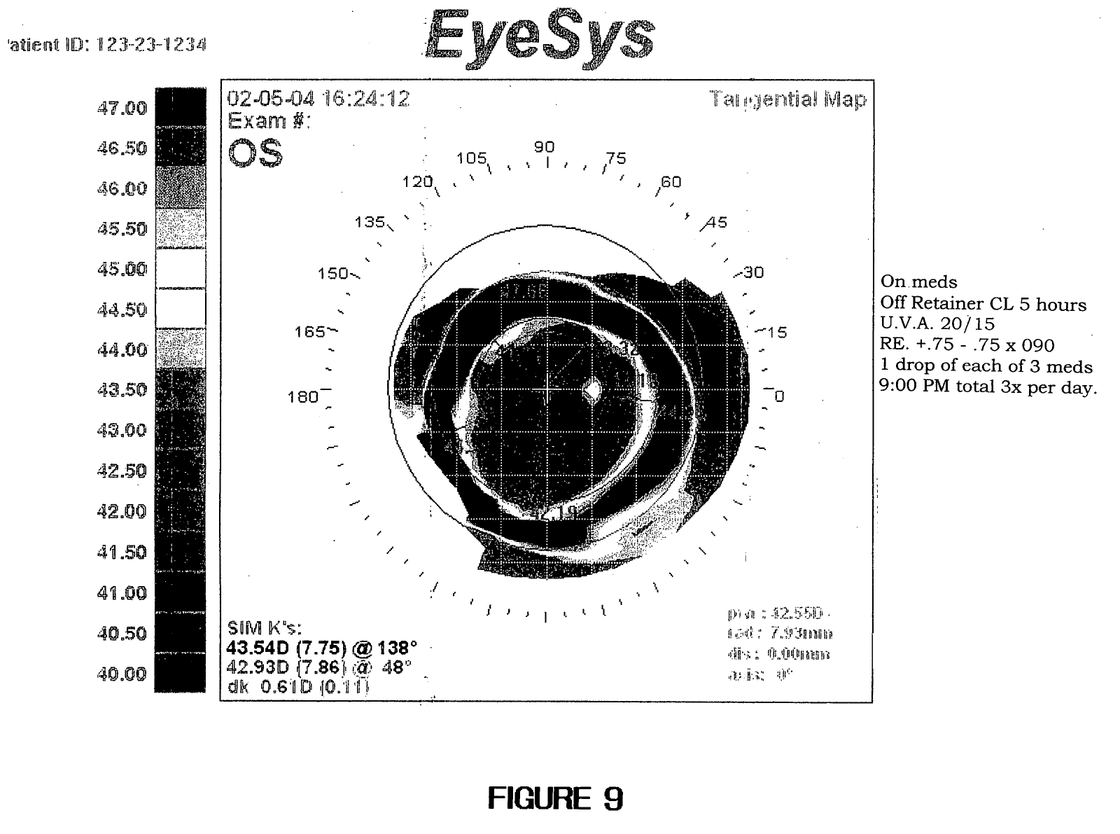

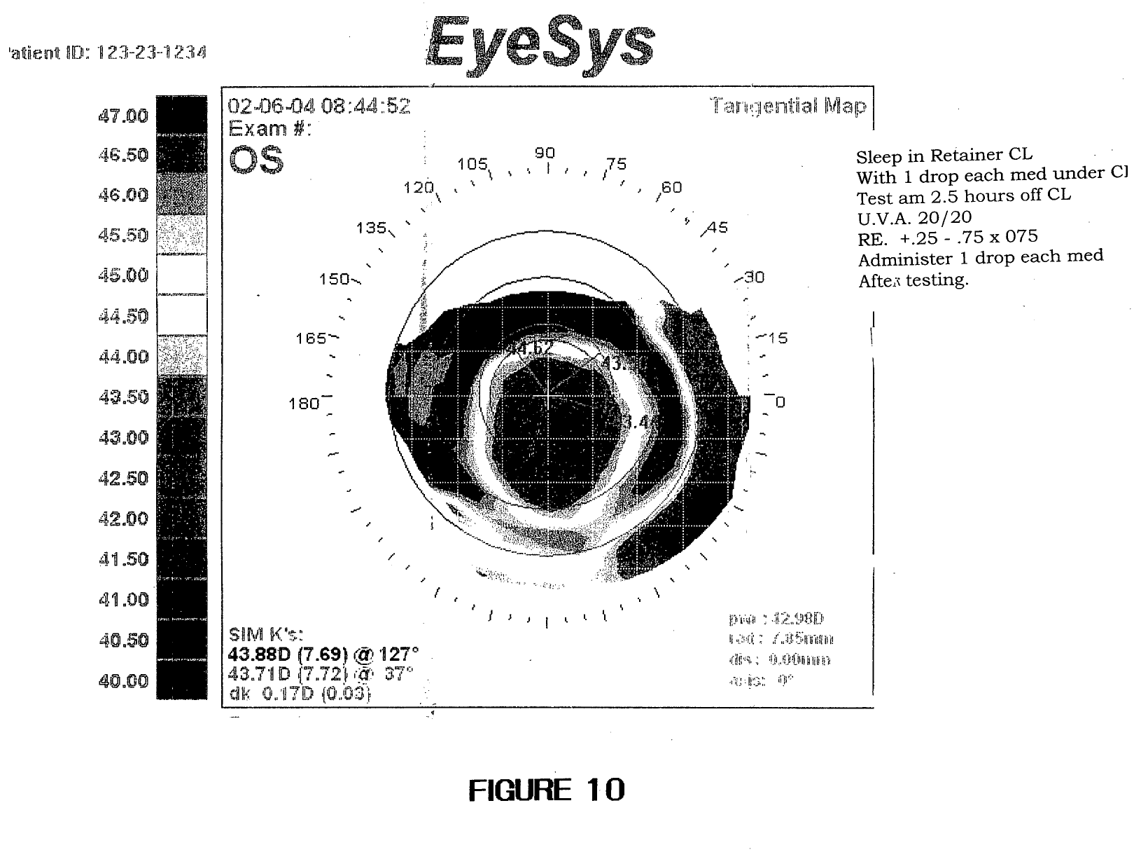

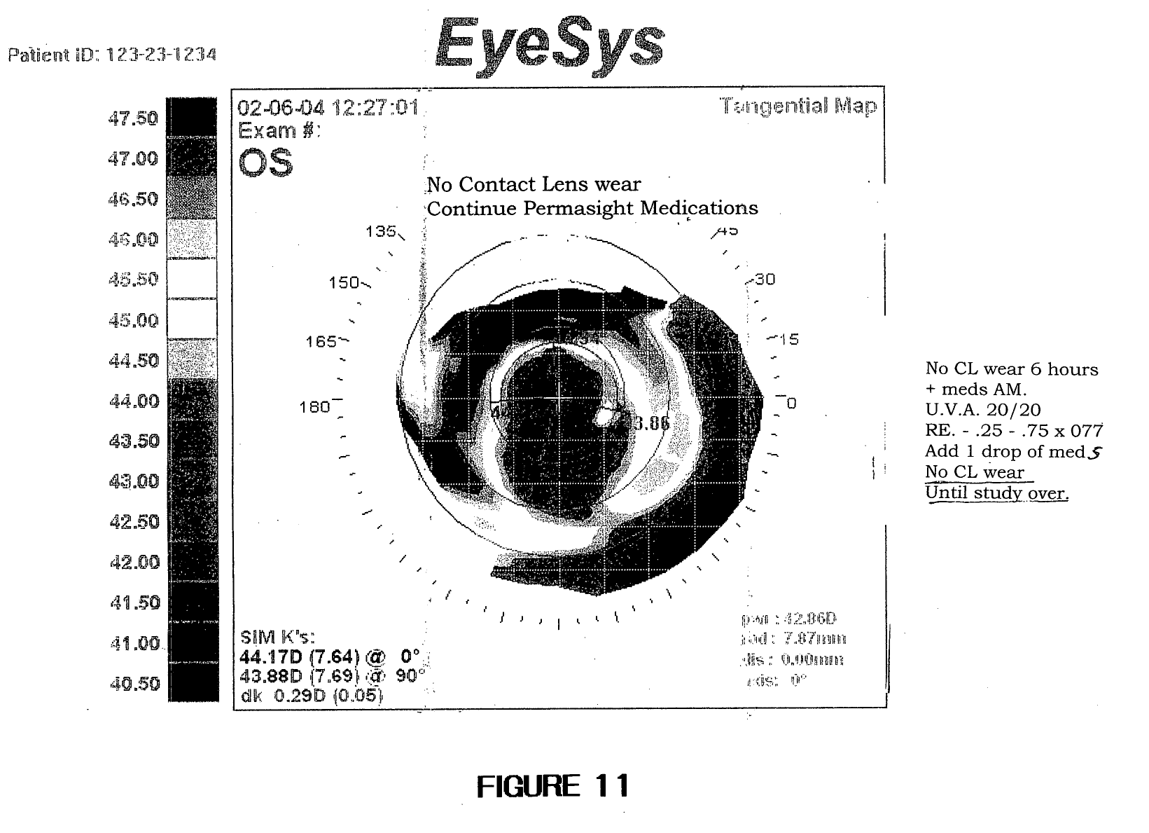

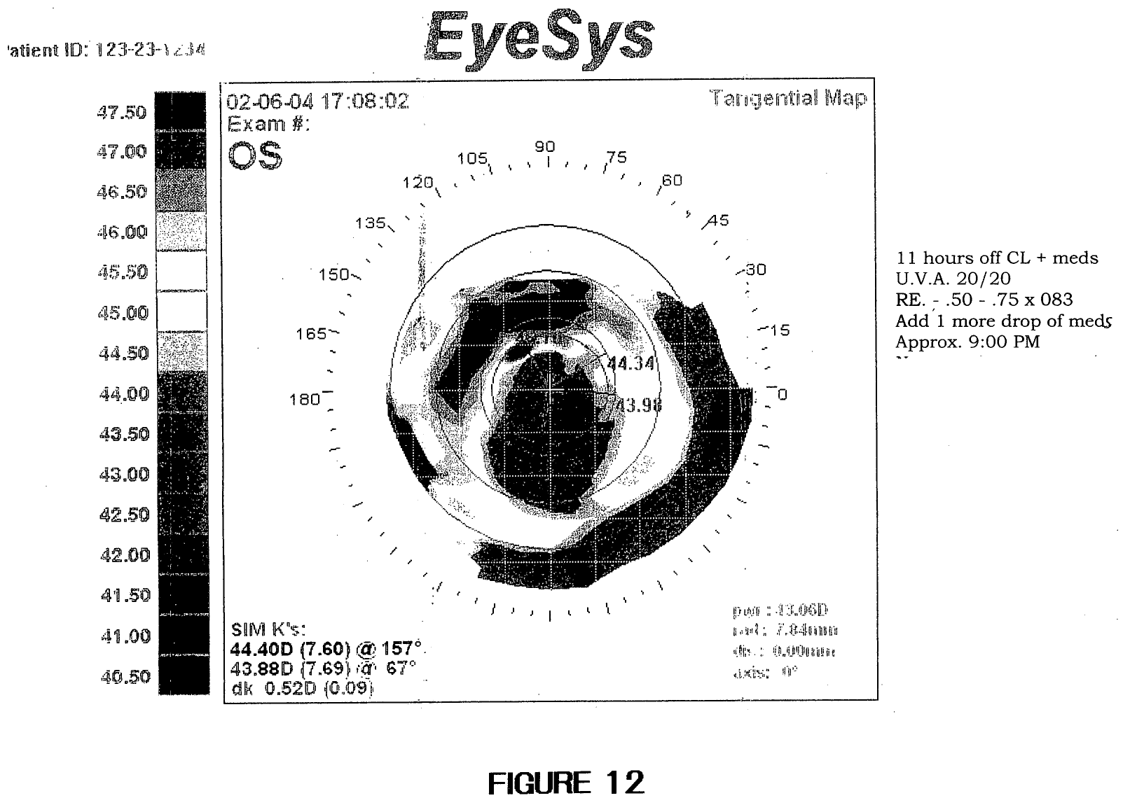

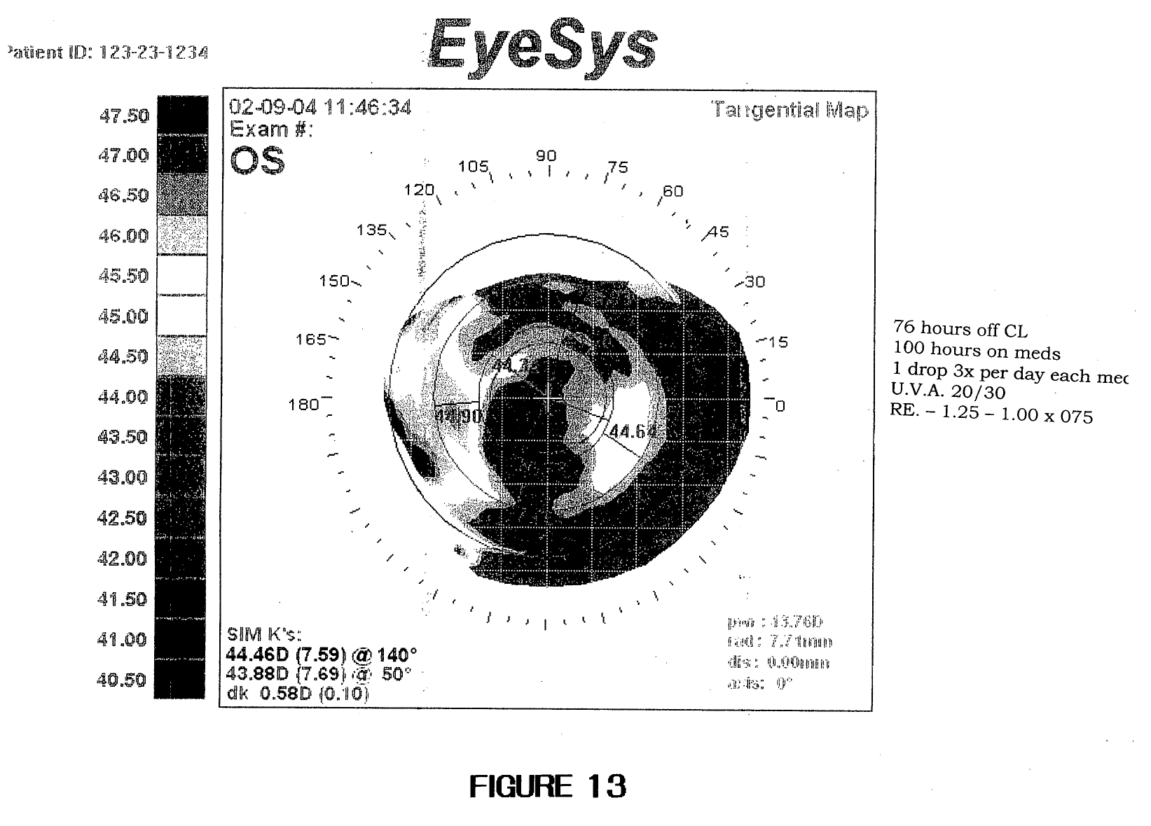

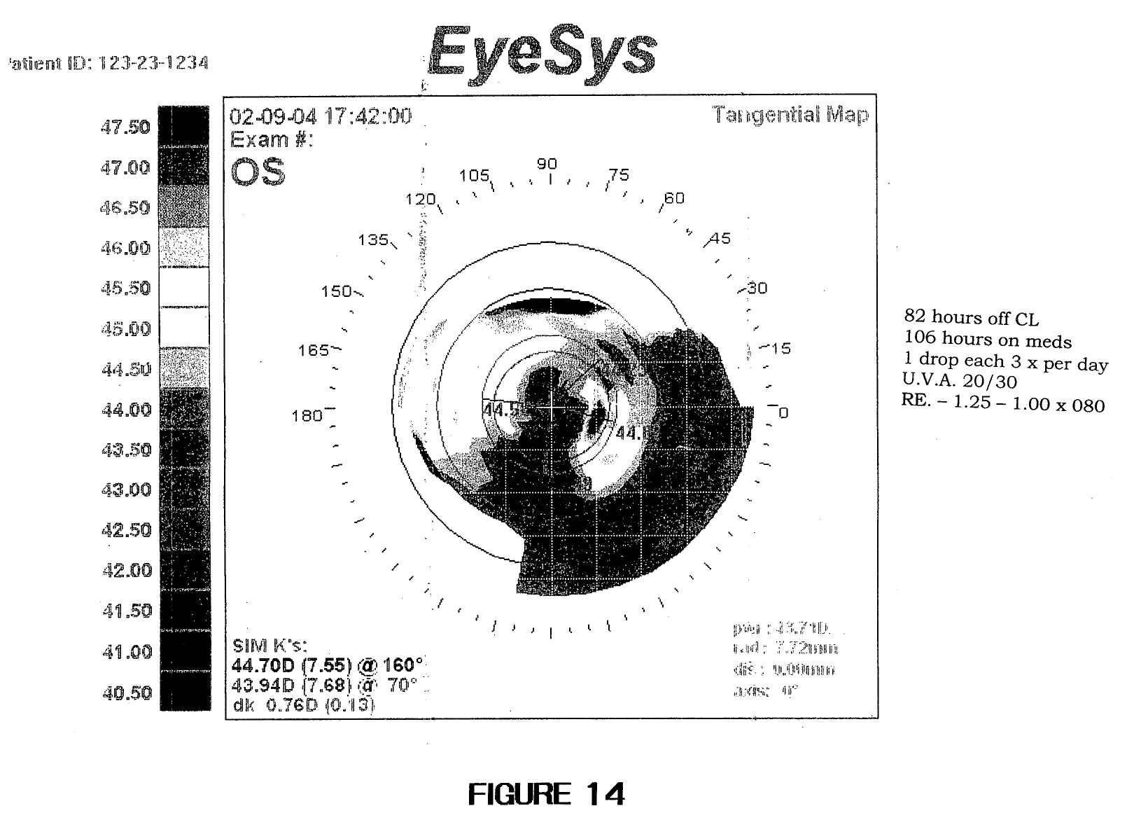

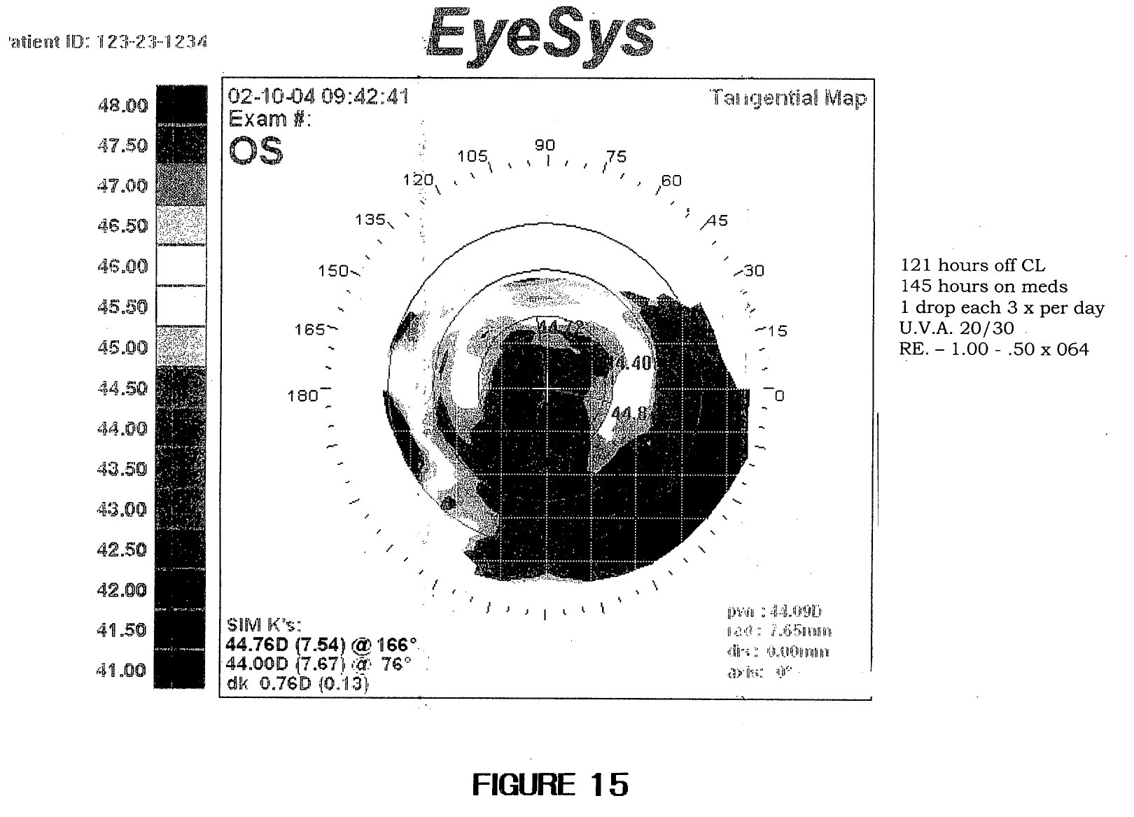

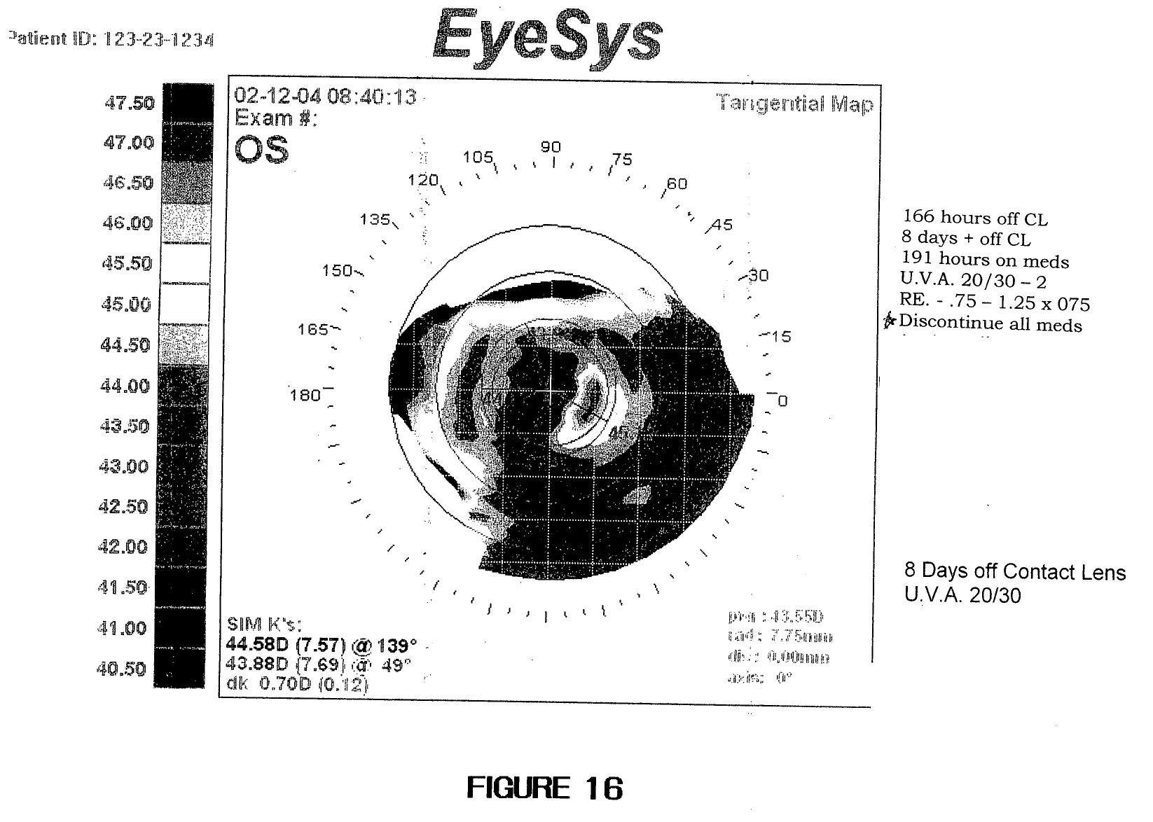

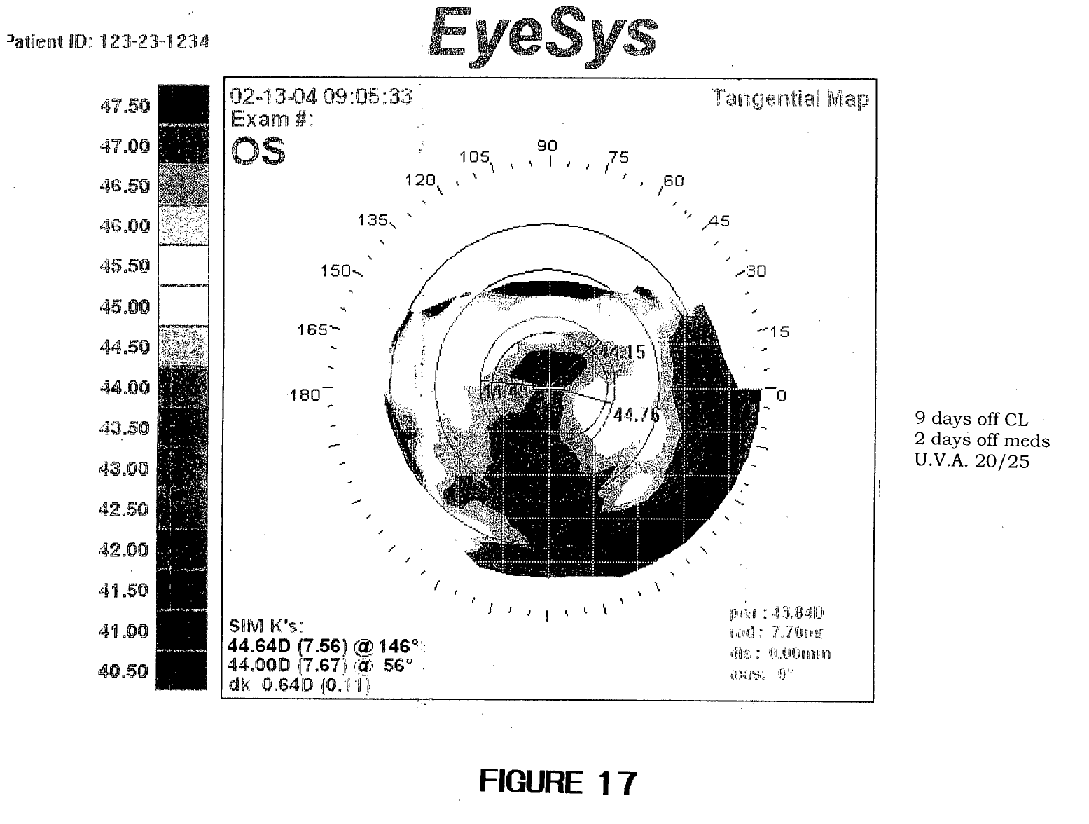

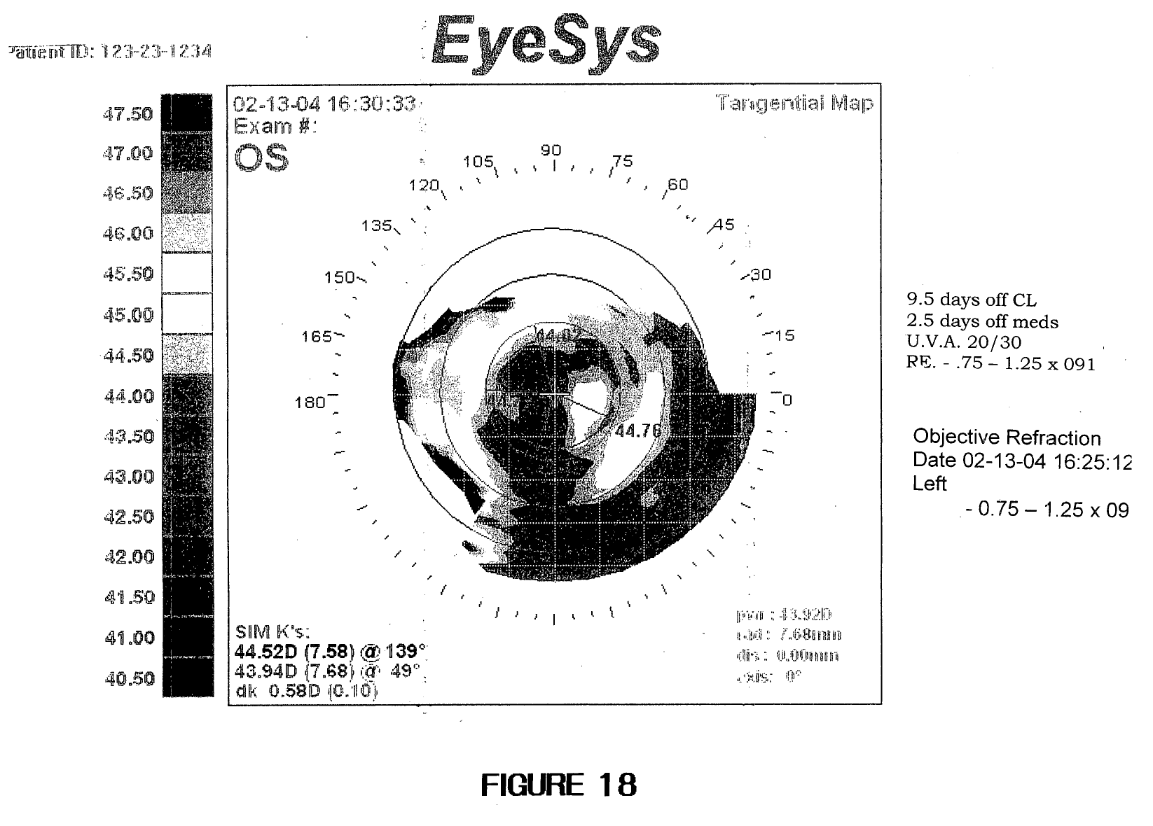

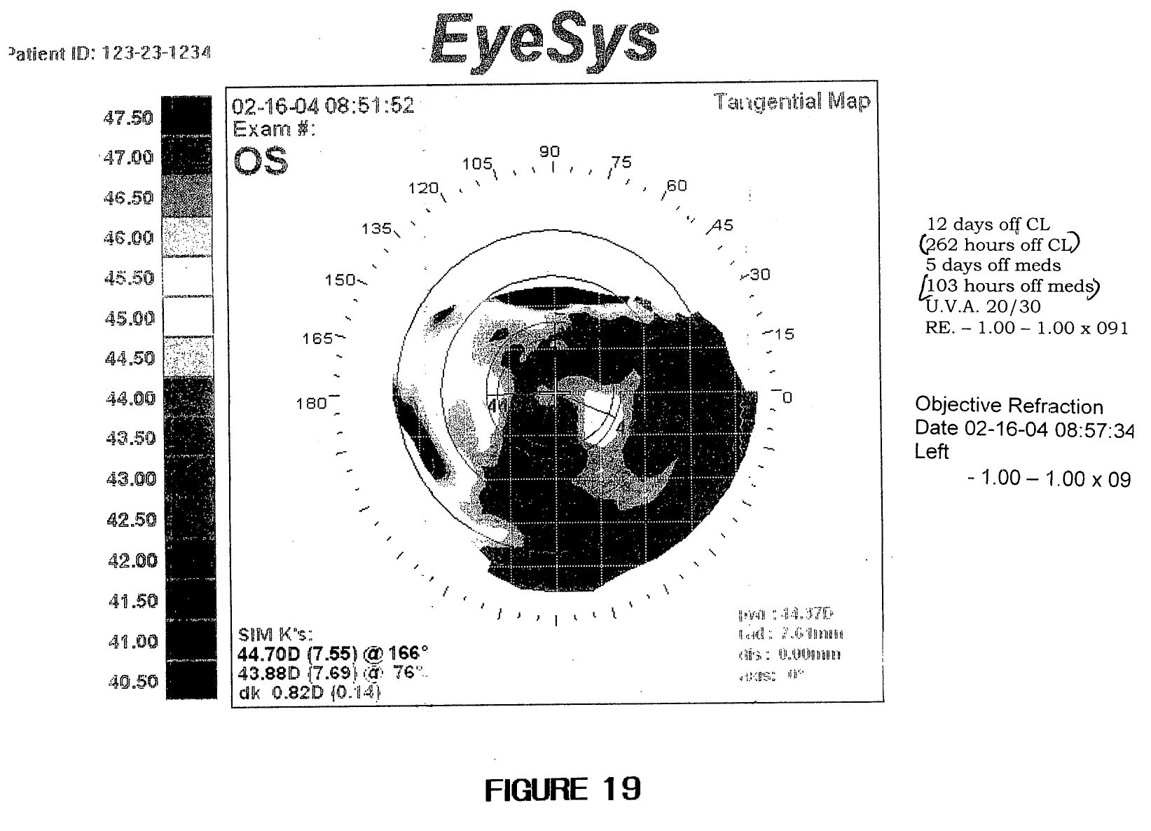

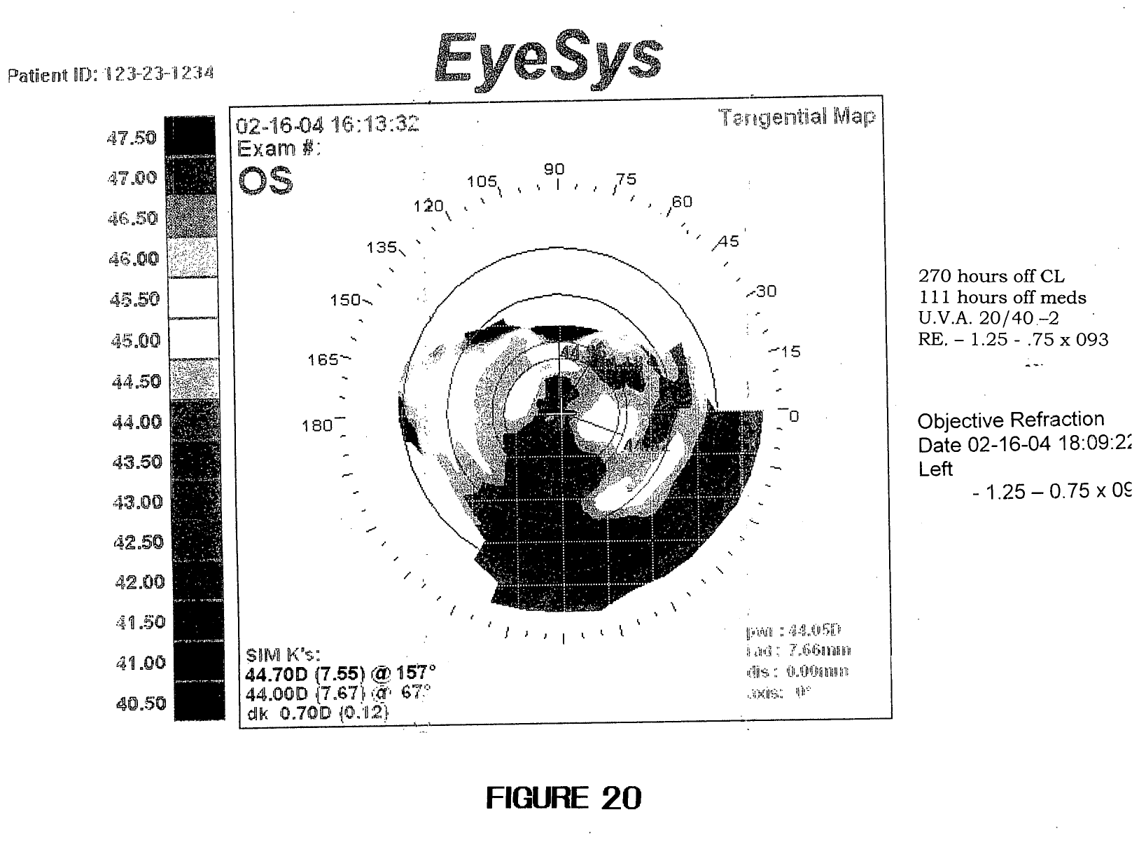

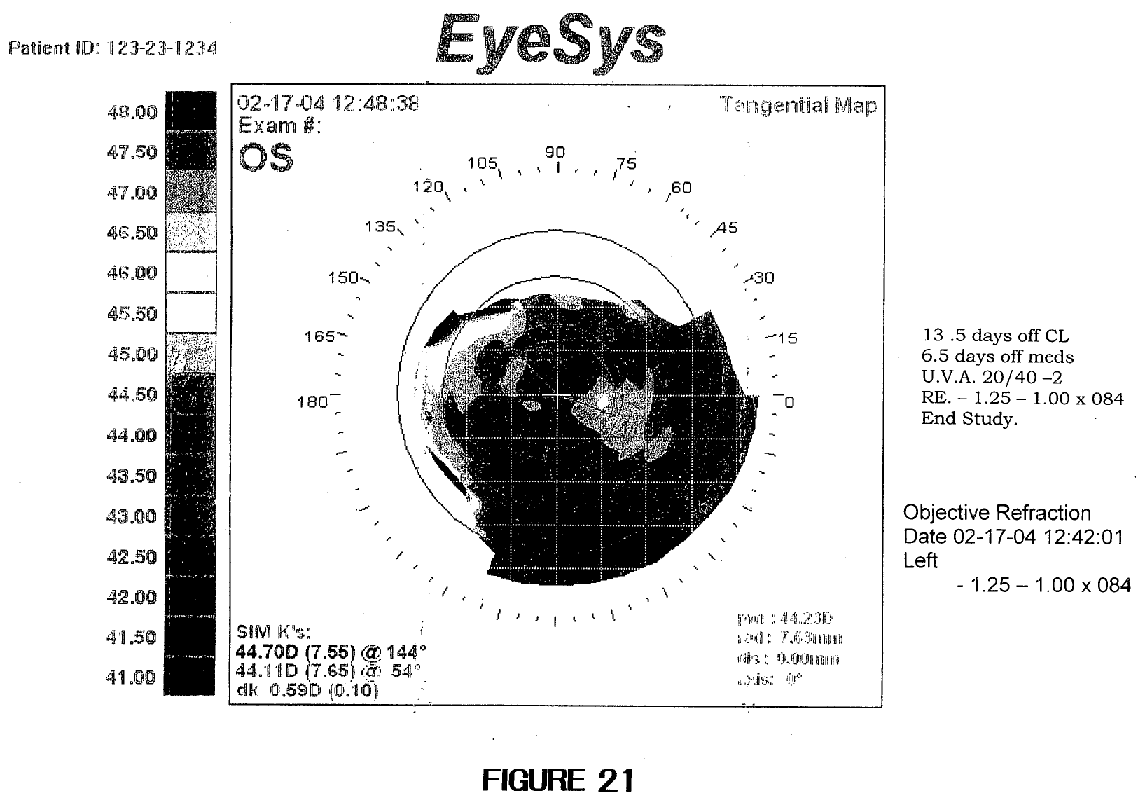

FIGS. 5-21 are representations of one patient's corneal topography changes over 12 days while using Permasight Ophthalmic Agents. These topographies represent corneal topography changes first using Ortho-K contact lens therapy to reshape cornea and then using three Permasight® Ophthalmic Agents (POA's).

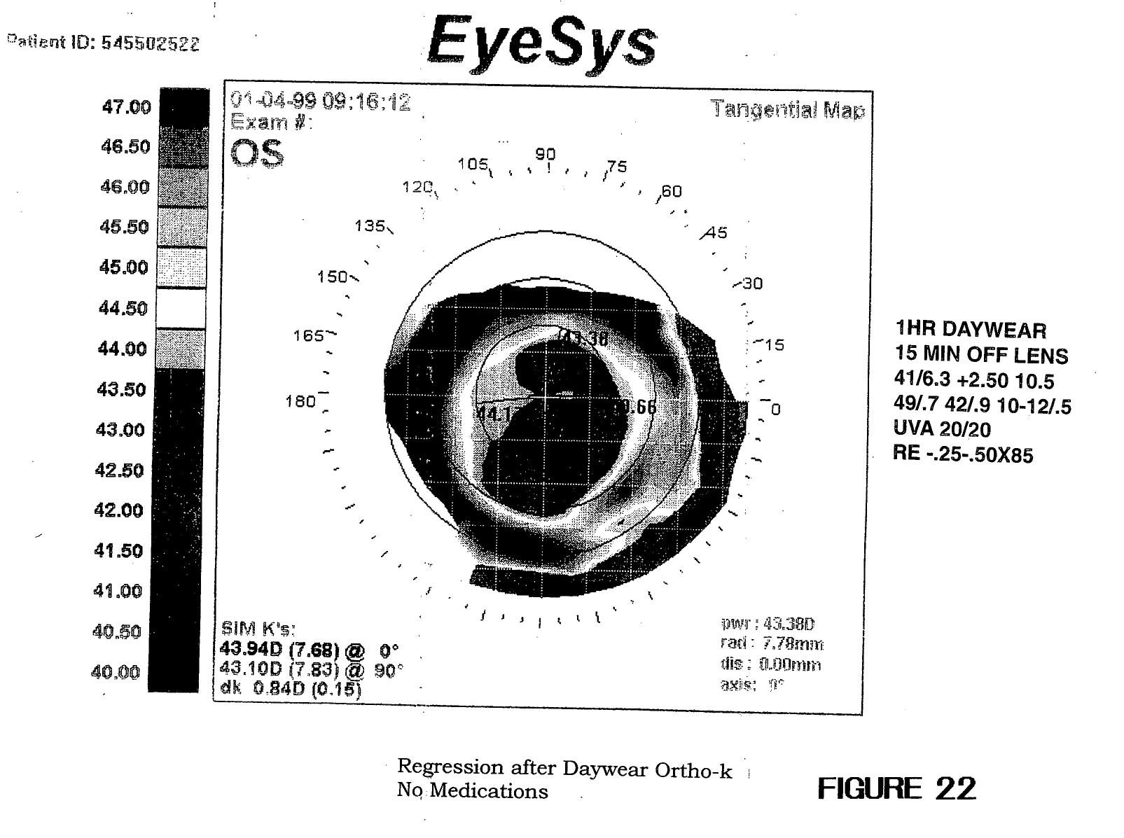

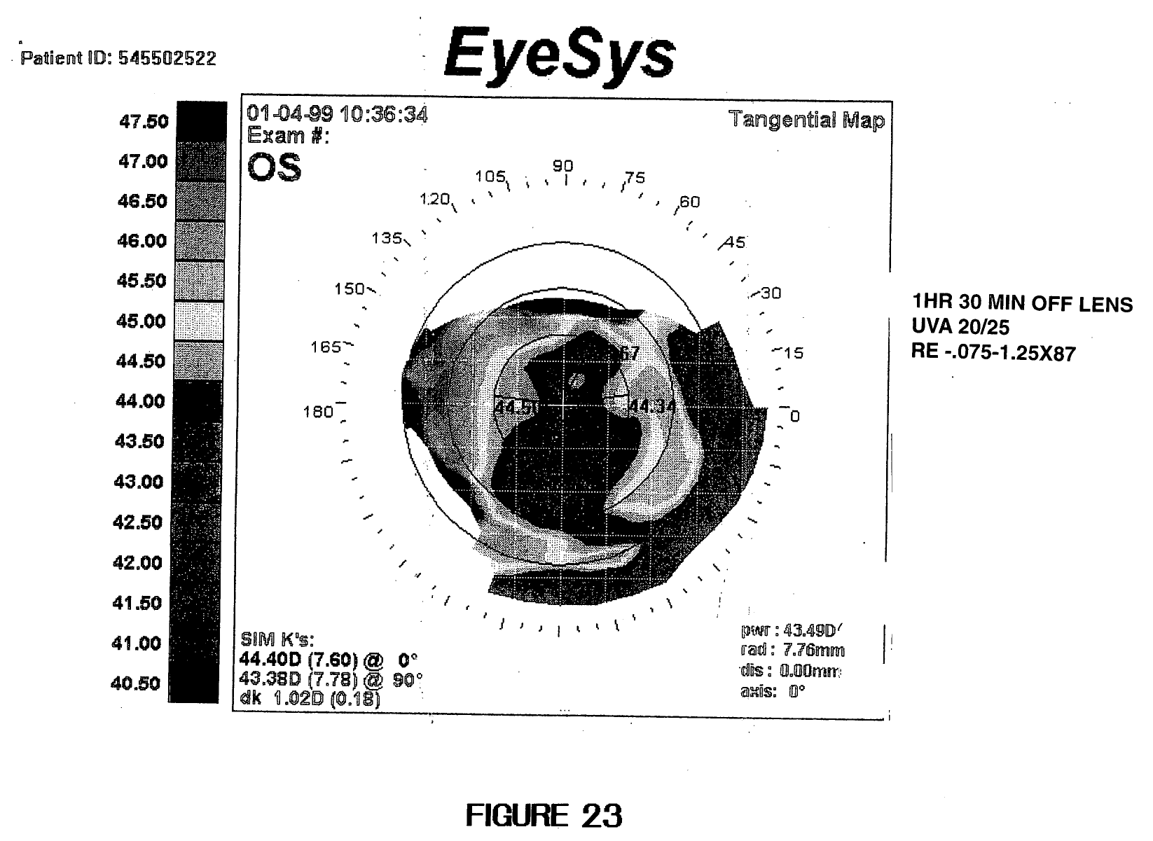

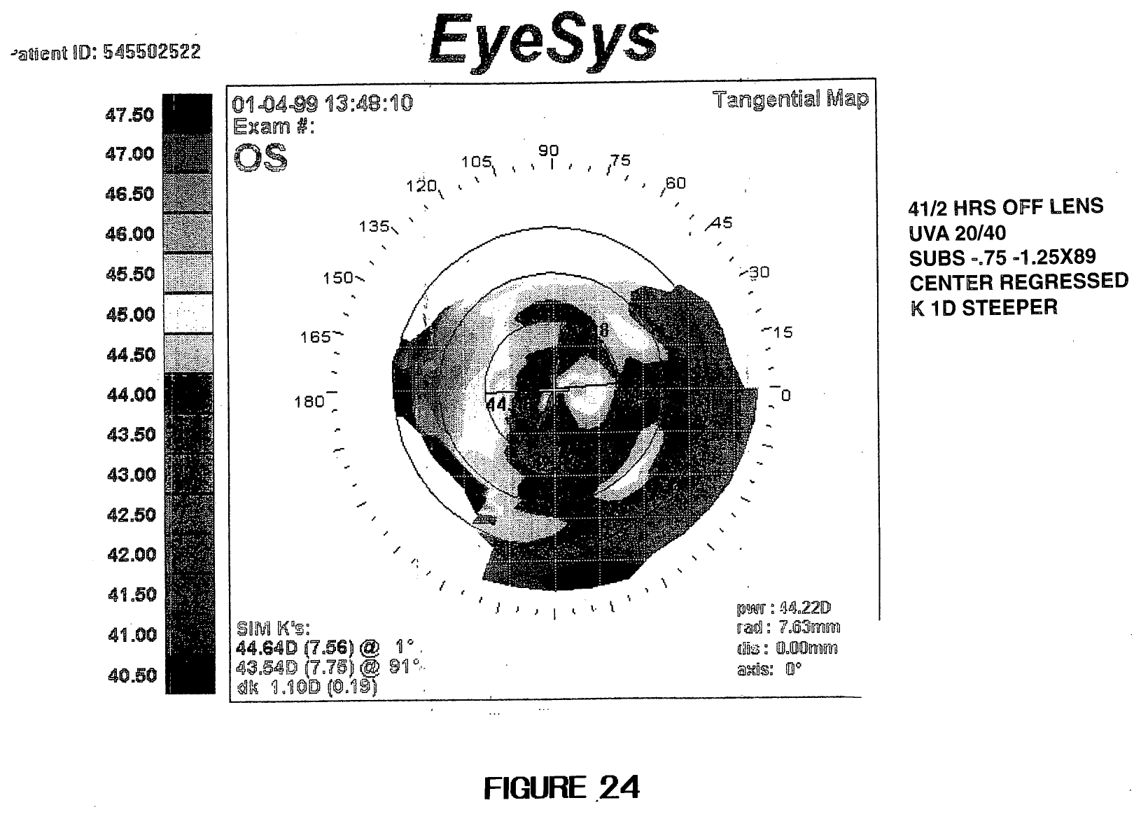

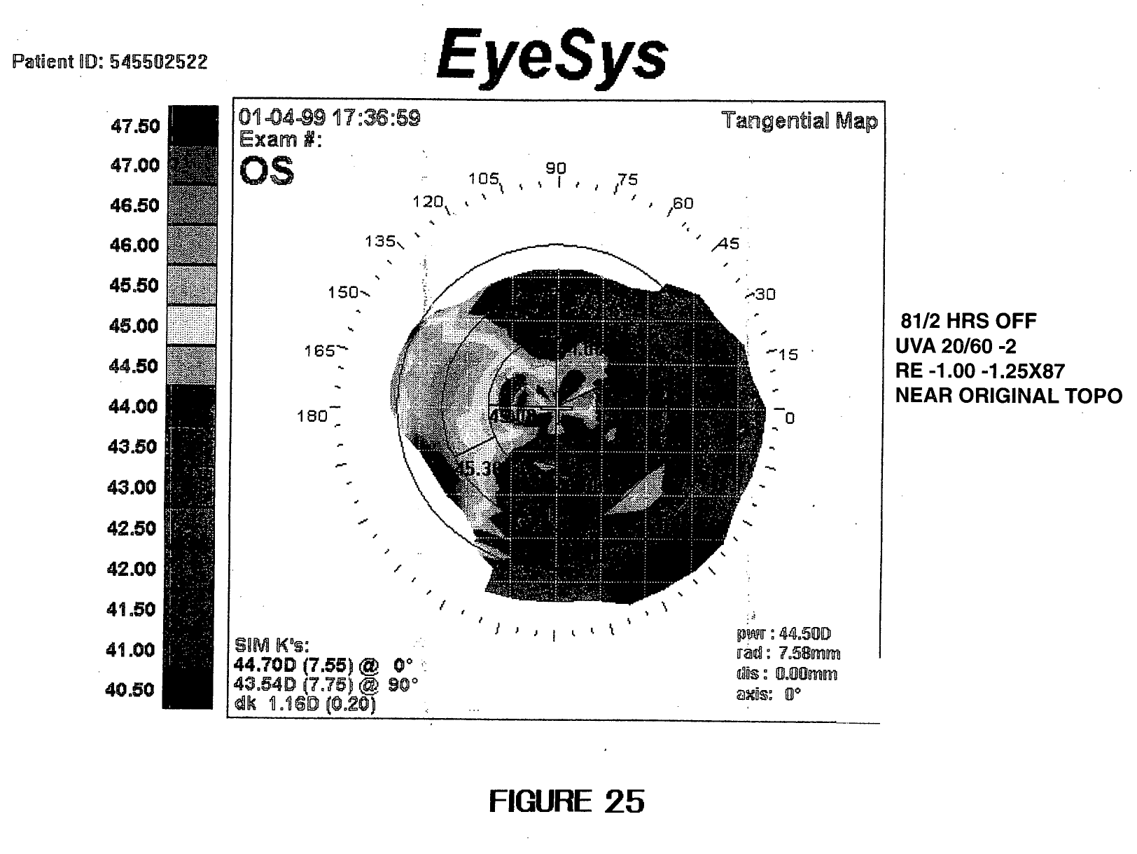

FIGS. 22-25 are corneal topographs of a nonstudy patient demonstrating corneal regression after day wear Ortho-K with no stabilizing agents being used.

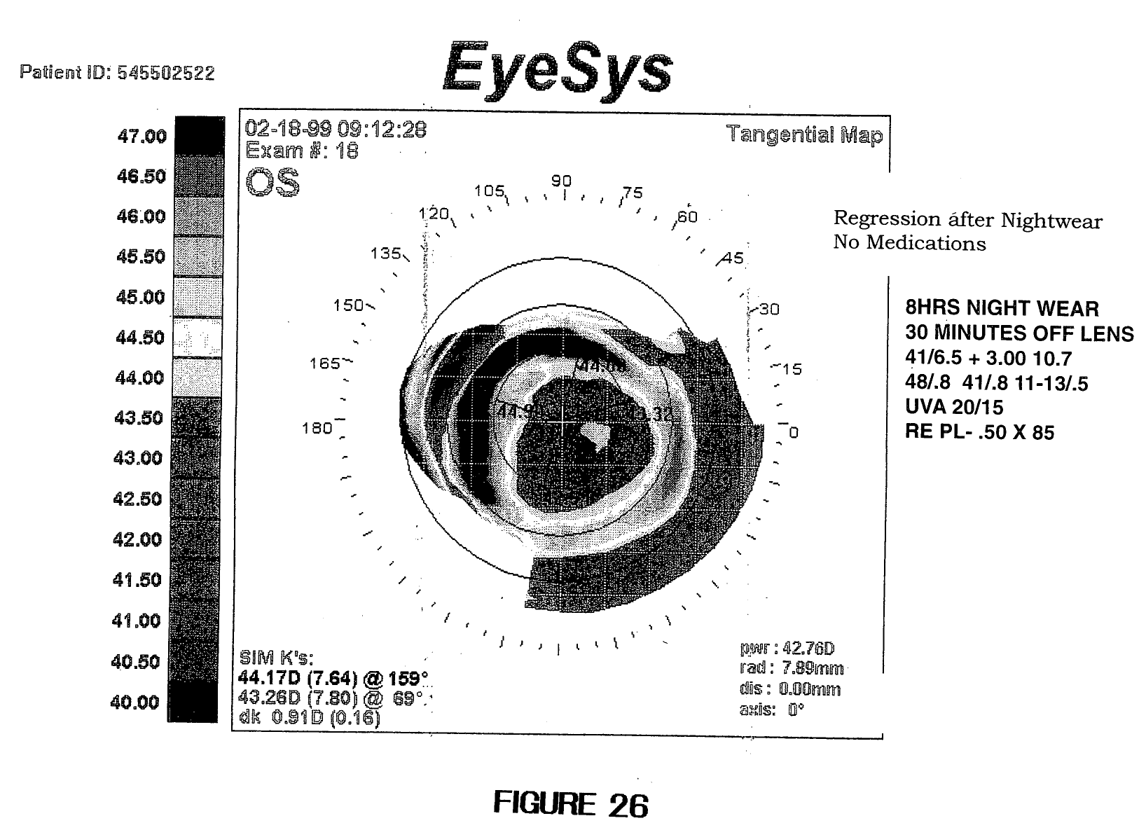

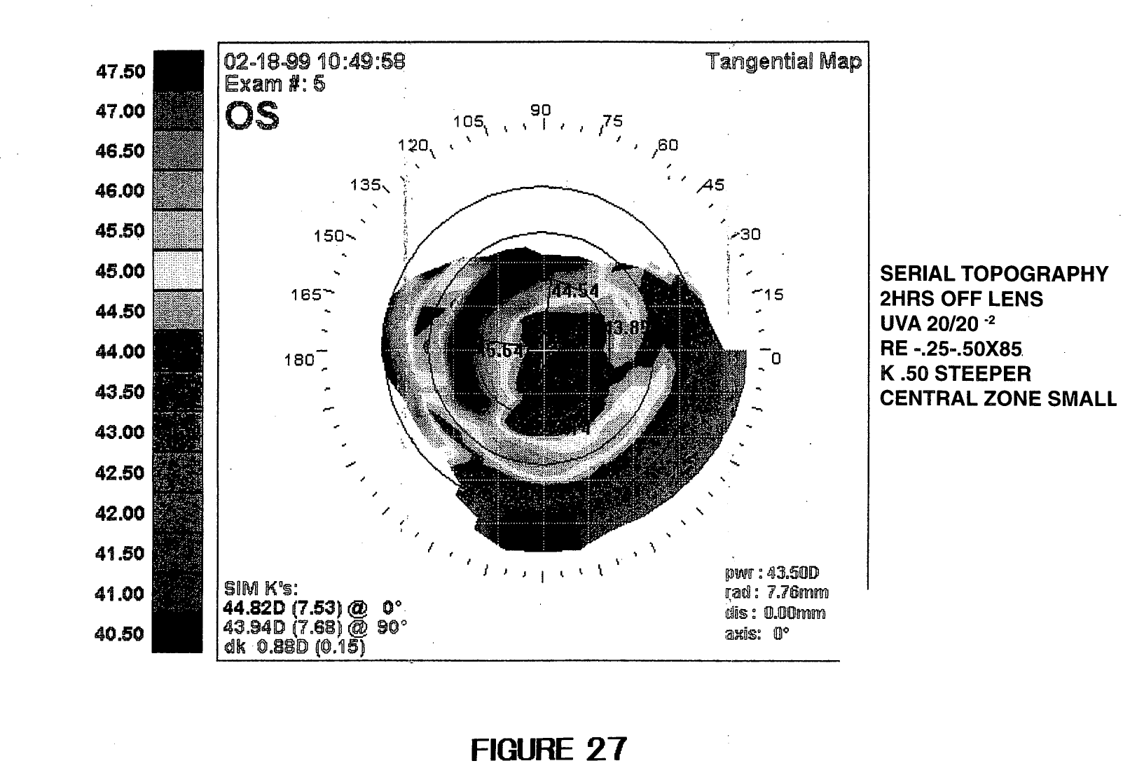

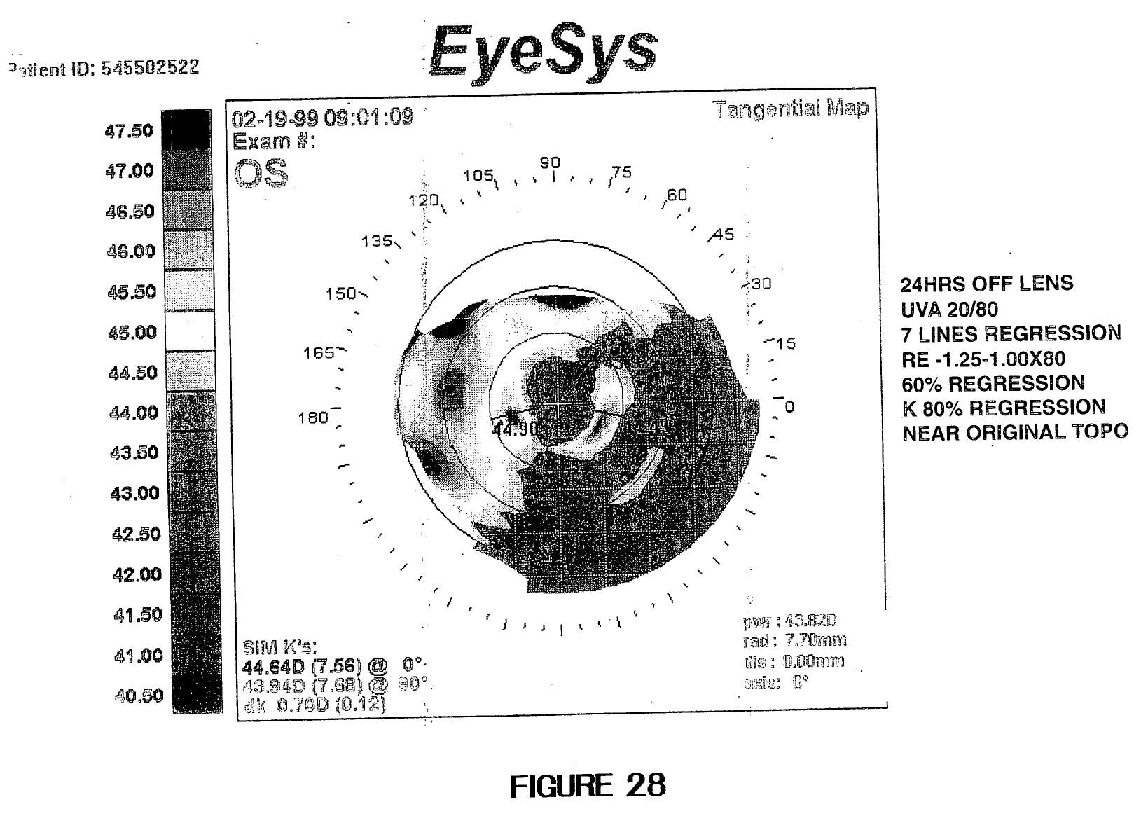

FIGS. 26-28 are corneal topographs of a second nonstudy patient demonstrating corneal regression after night wear Ortho-K with no stabilizing agents being used.

### BIBLIOGRAPHY

1. Hendrix, D. V., Ward, D. A. and Barnhill, M. A. 2001 Effects of antibiotics on morphologic characteristics and migration of canine corneal epithelial cells in tissue culture. Am J. Vet. Res. 62:1664-1669

2. Aragona, P., Tripodi, G., Spinella, R., Lagana, E., and Ferreri, G. 2000. The effects of the topical administration of non-steroidal anti-inflammatory drugs on corneal epithelium and corneal sensitivity in normal subjects. Eye 14:206-210

3. Kaji, Y., Amano, S., Oshika, T., Obata, H., Sakai, H., Shirasawa, E., Tsuru, T., and Yamashita, H. 2000. Effect of anti-inflammatory agents on corneal wound healing after sirface excimer laser keratotomy. J. Cataract Refract. Surg. 26:426-431

4. Matsumoto, S., and Stern, M. E. 2000. Effect of anti-infective ophthalmic solutions of corneal cells in vitro. Adv. Ther. 17:148-151

5. Stem, G. A., Schemmer, G. B., Farber, R. D., and Gorovoy, M. S.1983. Effect of topical antibiotic solutions on corneal epithelial wound healing. Arch. Opthalmol. 101:644-647

6. Nelson, J. D., Silverman, V., Lima, P. H., and Beckman, G. 1990. Corneal epithelial wound healing: a tissue culture assay on the effect of antibiotics. Curr. Eye Res. 9:277-285

7. Lin, C. P, Boehnke, M. 2000. Effect of fortified antibiotic solutions on corneal epithelial wound healing. Cornea.19:204-6.

8. Stem, G. A., Killingsworth, D. W. 1989. Complications of topical antimicrobial agents. Int. Ophthalmol. Clin. 29:137-42.

9. Gipson, I. K., Kiorpes, T. C., Brennan, S. J. 1984. Epithelial sheet movement:

effects of tunicamycin on migration and glycoprotein synthesis. Dev Biol.101 :212-20.

10. Nakamura, M., Nishida, T., Mishima, H., and Otori, T. 1993. Effects of antimicrobials on corneal epithelial migration. Curr. Eye Res.12:733-740

11. Kishimoto N, Uyama M, Fukushima I, Yamada K, Nishikawa M, Ohkuma H. 1993. The effect of corticosteroid on the repair of the retinal pigment epithelium.

Nippon Ganka Gakkai Zasshi.97:360-9.

12. Hashizume N, Saika S, Okada Y, Miyamoto T, Shimizu K, Ohnishi Y. 2001. Effects of antiinflammatory drugs on migration of the rabbit corneal epithelium. J Cataract Refract Surg. 27:1499-502.

13. Saika, S., Ohnishi, Y., Ooshima, A., Liu, C-Y., Kao, W. 2002. Epithelial Repair: roles of extracellular matrix. Cornea Suppl 1: S23-S29

14. Chandrasekher G, Ma X, Lallier T E, Bazan H E. 2002. Delay of corneal epithelial wound healing and induction of keratocyte apoptosis by platelet-activating factor. Invest Ophthalmol Vis Sci.43:1422-8.

15. McDonnell P J, Krause W, Glaser B M. 1988. In vitro inhibition of lens epithelial cell proliferation and migration. Ophthalmic Surg. 19:25-30.

16. Jung S, Fehr S, Harder-d'Heureuse J, Wiedenmann B, Dignass AU. 2001. Corticosteroids impair intestinal epithelial wound repair mechanisms in vitro. Scand J Gastroenterol. 36:963-70.

17. Nakamura M, Fujihara T, Mibu H, Hikida M. 1994. Arachidonic acid stimulates corneal epithelial migration. J Ocul Pharmacol. 10:453-9.

18. Savla U, Appel H J, Spom P H, Waters C M. 2001. Prostaglandin E(2) regulates wound closure in airway epithelium. Am J Physiol Lung Cell Mol Physiol.280:L421-31.

19. Schmassmann A. 1998. Mechanisms of ulcer healing and effects of nonsteroidal anti-inflammatory drugs. Am J Med. 104:43S-51S.

20. Pai, R, Szabo, I L, Giap, A Q, Kawanaka, H, Tarnawski, AS. 2001. Nonsteroidal anti-inflammatory drugs inhibit re-epithelialization of wounded gastric monolayers by interfering with actin, Src, FAK, and tensin signaling, Life Sciences 69:3055-3071

21. Mohajer B, Tarnawski A, Hoa N T, Tran D, Chen J, Park H, Ma T Y. 2002. Low dose ethanol potentiates indomethacin induced inhibition of wound re-epithelialization in duodenal monolayers. Life Sci. 70:3143-53.

22. Fenton, J I, Wolff, M S, Orth, M W, and Hord, N G. 2002. Membrane-type matrix metalloproteinases mediate curcumin-induced cell migration in non-tumorigenic colon epithelial cells differing in Apc genotype. Carcinogenesis 23:1065-1070.

1. A method for stabilizing changes in corneal curvature in an eye, comprising administering to an eye having a changed corneal curvature, a composition comprising at least one stabilizing ophthalmic agent, in an amount effective to substantially stabilize the changed curvature of the cornea.

2. The method of claim 1, wherein the corneal curvature is changed to stabilize the improved refractive error and unaided visual acuity of the eye.

3. The method of claim 1, wherein said ophthalmic agent prevents remigration of epithelial cells to the changed area of the cornea.

4. The method of claim 1, wherein said cornea is reshaped to correct myopia, astigmatism and/or hyperopia.

5. The method of claim 1, wherein the corneal curvature of the eye is reshaped by orthokeratology, corneal refractive therapy and/or refractive surgery treatment.

6. The method of claim 1, wherein the stabilizing ophthalmic agent comprises one or more compounds selected from the group consisting of compounds that slow down wound healing, compounds that ameliorate autoimmune diseases, compounds that change the elasticity of the cornea, compounds that change the rate of epithelial cell migration, and compounds that change the distribution of cornea cells.

7. The method of claim 1, wherein the ophthalmic agent comprises one or more compounds selected from the group consisting of non steroidal anti-inflammatory compounds, corticosteroids, antihistamines, antibiotics, mast cell stabilizers and metalloproteinases.

8. The method of claim 1, wherein the ophthalmic agent is a composition containing one or more of Acular®, Patanol®, Ocuflox®, Alrex®, Alamast®, Lotemax and Tobramycin.

9. The method as in claim 1, wherein the ophthalmic agent comprises a tear reinforcement agent such as Restatis®.

10. The method as in claim 1, wherein the ophthalmic agent is used in combination with the ocular anesthetic proparacaine.

11. The method as in claim 1, wherein the ophthalmic agent is used in combination with the preservative Thimerosol or Benzalkonium chloride.

12. The method of claim 1, wherein the ophthalmic agent is administered topically.

13. The method of claim 12, wherein the ophthalmic agent is administered topically in a drop form, gel form or embedded in a contact lens.

14. A method for stabilizing altered corneal curvature in an eye comprising:
a. reshaping the corneal curvature; and
b. administering a composition comprising at least one stabilizing ophthalmic agent, in an amount effective to stabilize the curvature of the reshaped cornea.

15. A composition for stabilizing the curvature of a reshaped cornea, comprising one or more agents selected from the group consisting of stabilizing ophthalmical compounds that slow down wound healing, compounds that ameliorate autoimmune diseases, compounds that change the elasticity of the cornea, compounds that change the rate of epithelial cell migration, and compounds that change the distribution of cornea cells, in an ophthalmically compatible solution.

16. The composition of claim 15, wherein the stabilizing ophthalmical agent comprises one or more compounds selected from the group consisting of non-steroidal anti-inflammatory agents, corticosteroids, antihistamines, antibiotics, mast cell stabilizers and metalloproteinases.

17. The composition of claim 16, comprising one or more of Acular®, Patanol® and Ocuflox®.

18. The composition of claim 15, wherein the ophthalmic agent is a composition containing one or more of Acular®, Patanol®, Ocuflox®, Alrex®, Alamast®, Lotemax and Tobramycin.

19. The composition of claim 15, wherein the ophthalmic agent comprises a tear reinforcement agent such as Restasis®.

20. The composition of claim 16, wherein the ophthalmic agent, is in combination with the ocular anesthetic proparacaine.

21. The composition of claim 16, wherein the ophthalmic compatible agent is used in combination with the preservative Thimerosol or Benzalkonium chloride.

22. A method of stabilizing altered corneal curvature in an eye comprising administering the composition of claim 15, 16, 17, 18, 19, 20 or 21, to an eye having altered corneal curvature, in an amount effective to stabilize the altered curvature of the cornea.

23. The method of claim 1, 7, 15 or 16, wherein the ophthalmic agent is a compound or combination of compounds, that stabilize corneal curvature, refractive error or unaided visual acuity improvement, retard epithelial cell migration and is topically administered.

24. A kit for stabilizing altered corneal curvature in an eye having altered corneal curvature, comprising the composition of claim 15, 16, 17, 18, 19, 20 or 21.

25. A method for stabilizing changes in corneal curvature in an eye, comprising administering to an eye having a changed corneal curvature, a composition comprising more than one stabilizing ophthalmic agent, in an amount effective to substantially stabilize the changed curvature of the cornea.

26. A method of stabilizing changes in corneal curvature in an eye, comprising:
a. reshaping the corneal curvature; and
b. administering a composition comprising more than one stabilizing ophthalmic agent, in an amount effective to stabilize the curvature of the reshaped cornea.

27. The method of claim 25 or 26, wherein said ophthalmic agent is selected from the group consisting of one or more compounds selected from the group consisting of non-steroidal anti-inflammatory agents, corticosteroids, antihistamines, antibiotics, mast cell stabilizers and metalloproteinases.

28. The method of claim 25 or 26, wherein said ophthalmic agent is selected from the group consisting of Acular®, Patanol®, Ocuflox®, Alrex®, Alamast®, Lotemax and Tobramycin.

29. The method of claim 25 or 26, wherein the ophthalmic agent comprises a tear reinforcement agent such as Restasis®.

30. The method of claim 25 or 26, wherein the ophthalmic agent is in combination with the ocular anesthetic proparacaine.

31. The composition of claim 25 or 26, wherein the ophthalmic agent is used in combination with a the preservative Thimerosol or Benzalkonium chloride.
